SEQUENCE LISTING (27 sequences)

SEQ ID NO: 1 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETERISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 96 aa, Artificial Sequence
  Synthetic amino acid sequence COP-5
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 3 - protein, 96 aa, Artificial Sequence
  Synthetic amino acid sequence COP-7
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 4 - protein, 102 aa, Artificial Sequence
  OP-X
CXXHELYVXFXDLGWXDWXIAPXGYXAYYCEGECXFPLXSXMNATNHAIXQXLVHXXXPXXVPKXCCAPTXLXAXSVLYXDXSXNVXLXKXRNMVVXACGCH

SEQ ID NO: 5 - protein, 97 aa, Artificial Sequence
  Generic Sequence 7
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 102 aa, Artificial Sequence
  Generic Sequence 8
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 7 - protein, 97 aa, Artificial Sequence
  Generic Sequence 9
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 102 aa, Artificial Sequence
  Generic Sequence 10
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 9 - protein, 5 aa, Artificial Sequence
  Consensus Sequence
CXXXX

SEQ ID NO: 10 - DNA, 1822 bp, Homo sapiens
  CDS (49)..(1341)
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 11 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 12 - DNA, 3151 bp, Mus musculus
ggaggctgggcggttccgactgcctgacgccagcgcgaccagatcaatccaggctcggggagcgagcgggcgcaccaaggcgaggccggggccgaggcgcggggacggcggcccggagctaagcagagcgcggggacggcggccgggagcggatcggagcacgggacccggcgccgggtctcgtgcgtccctgcggatgggcgcgccgccgagccggcgctaactgggatcctcgctggaacaggagggacagtattttctacctttccaaaccgcagaccaagaagctaaggagaatctatgtaaatatactgaaatctctgttggctctgcgcccaacaccccggagctggcacctcaccctgtctgaggagcgtgtagaactagaccagccgctatgaatgtgaccagcttgttttcattcacaagtccagctgtgaagagactccttgggtggaaacagggcgatgaagaagagaaatgggcagagaaagctgtggacgctttggtgaagaaactgaagaagaagaaaggggccatggaagagctggagaaggccctgagctgccctggacagccgagtaactgcgtcaccattcctcgctccctggatggcaggttgcaggtgtcccaccggaagggactacctcatgtcatttattgccgtgtgtggcgctggcccgacctccagagccaccatgaactgaagcctctggaatgctgtgagttcccatttggttccaagcagaaggaggtctgcatcaacccctaccactataagcgagtggagagcccggttctcccgccggtgctggttccgaggcacagcgagtacaatcctcagcacagccttctggctcagttccgcaacctgggacaaaatgagcctcacatgccactgaacgccacgttcccagactctttccagcagcccaacagccacccgttcccccactcccccaacagcagctaccccaactctcctggcggcagcagcagcacctaccctcactccccaaccagctcagacccgggcagcccttttcagatgccagctgacacacccccacctgcttacctgcctcctgaagaccccatggcccaggatggctctcagcccatggacacgaacatgatggcgcctccactgcccgctgaaatcagcagaggagatgttcaggcagttgcttacgaggaaccaaaacactggtgctctattgtgtactatgagctcaacaaccgtgtgggtgaagcgttccacgcctcgtccaccagcgtgctggtggatggtttcacagatccgtccaacaataagaaccgcttctgccttggcttgctctccaacgttaaccggaattccactattgaaaacaccaggcgacatattgggaaaggagtccacctttattacgttggaggagaggtgtatgcggaatgcctcagtgacagcagcatcttcgtgcagagccggaactgcaactaccaccacggctttcaccccaccaccgtctgcaagatccccagcgggtgcagcttgaaaatcttcaacaaccaagagtttgctcagctactggcgcagtctgtgaaccacgggttcgagaccgtgtatgaactcaccaaaatgtgcactattcggatgagcttcgtgaagggttggggagccgaataccaccggcaggatgttaccagcaccccctgctggattgagatccatctgcatggccctctccagtggctggataaggttctgacccagatgggctcaccccacaatcctatttcatccgtgtcttaaaagacctgtggcttccgtctcttgcaaactatcgagccttgcatgtacttgaaggatggacaagtcagacaggatggagacctgacgaaggagccacgataatacttgacctctgtgaccaactattggattgagaaactgacaagccttggttgatagcaagaaccctttcagtttacattgtgacattctgttgtaaaaatcaactaaaatgctgactttcagcaggacttttgtgtatagttaaaaaaaaaagagatggccaagccagggacaaattatctattaggaaaaaagaaaaaaatgattgtaatcaatccttttgtgtggggtgttggcagaaggttggcgctgatagtctttctgaagtgggctttcatcaggctcagagcccacgttgaatcatcttctcatgggttttcttaatattttaaaactacttgtttagaaatgaatgggttttttgtttgtttttaaagtacaggttaatcgttatgacatgcatagtaatctttctgaaactgtatgctggctgtattactgtcagaatgatggcaggcatatgctctttgctaaatatgtatatacagaatatttggaggttatgaatagtctaaatggctagtgggtttacagagtatctgaggggcggggtcgggaagaaaacgacggctgcaaatgtagactataccgtaaagctcagcttgctgccttaaacagacaagctggtgtctgaatttgctgtgtttcagtttttgtagagttttatctgacttcttttcttctgtcttatccgctccacggcacagttaagcagctggttaattcctctaactgtgagagcagatgagtaattccttctgttcgcaaatcaactggcttcgtgtttcagtacccagtatatgaaaagcttgaattgaatgagcagtttttatggagtttacagtacagacataggctttgatttccaaataaattgtttgccaaacctggtaactctgttcattattcgcaggattaaagatctctctattggaatccatttcaaaggttgttttttttgtttttgtttttgttttttgttttattttgatttgtttttttttgtactatttggtttcttttcttctgttaatttttttattctcctttgctcttatacagcgagtacttttattccaacactagcagggtttttctctactggaaatttttaaataaaacctgtcattattgcttactttgattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatttcatctgtgtcttaa

SEQ ID NO: 13 - protein, 465 aa, Mus musculus
MNVTSLFSFTSPAVKRLLGWKQGDEEEKWAEKAVDALVKKLKKKKGAMEELEKALSCPGQPSNCVTIPRSLDGRLQVSHRKGLPHVIYCRVWRWPDLQSHHELKPLECCEFPFGSKQKEVCINPYHYKRVESPVLPPVLVPRHSEYNPQHSLLAQFRNLGQNEPHMPLNATFPDSFQQPNSHPFPHSPNSSYPNSPGGSSSTYPHSPTSSDPGSPFQMPADTPPPAYLPPEDPMAQDGSQPMDTNMMAPPLPAEISRGDVQAVAYEEPKHWCSIVYYELNNRVGEAFHASSTSVLVDGFTDPSNNKNRFCLGLLSNVNRNSTIENTRRHIGKGVHLYYVGGEVYAECLSDSSIFVQSRNCNYHHGFHPTTVCKIPSGCSLKIFNNQEFAQLLAQSVNHGFETVYELTKMCTIRMSFVKGWGAEYHRQDVTSTPCWIEIHLHGPLQWLDKVLTQMGSPHNPISSVS

SEQ ID NO: 14 - DNA, 2449 bp, Mus musculus
ggttaaaataactatctgagatttgttttgctgttgttgttgtttaaggaaaattaaggtagtaccatatcttaaatcattgcaacaagaggcagtattgctacttataaaagtaaataatagtgtataaaattgtgtttcaaccgaatcttactggcatctttctctctttcttggaaacactccatgaaacaatagatgcagtagatcaggatgatgaggacgggaatgggggcactactacactactatactactacactctaggattcgaggctgcatgcagagttaacaacagtcagctgactgtttacctgaaagactggcatagaataggaaaatttggtgccaagtgcataaaaataagcaaatgaaaagacattaattctgggtagatttaccgggctttttctgagtgtggattgttacctttggtaagaaaatgtcgtccatcttgccattcactccgccagtggtgaagagacttctgggatggaaaaaatcagccggtgggtctggaggagcaggtggtggagagcagaatggacaggaagaaaagtggtgtgaaaaagcagtgaaaagtctggtgaaaaagctaaagaaaacaggacggttagatgagcttgagaaagccatcaccactcagaattgcaatactaaatgtgtcaccataccaagcacttgctctgaaatttggggactgagtacagcaaatacggtagatcagtgggacacaacaggcctttacagcttctctgaacaaaccaggtctcttgatggccgtcttcaggtttcacaccggaaagggttgccacatgttatatattgccggctctggcgctggccggaccttcacagtcatcatgagctcaaggcaatcgaaaactgcgaatatgcttttaatctgaaaaaagatgaagtgtgtgtaaatccgtaccactaccagagagttgagaccccagtcttgcctccagtcttagtgcctcggcacacggagattctaacagaactgccacccctggatgactacacccactccattccagaaaacacaaatttcccagcaggaattgagccacagagtaattacatcccagaaacaccaccacctggatatatcagtgaagatggagaaacaagtgaccaacagttgaaccaaagtatggacacaggctctccggctgaactgtctcctactactctctctcctgttaatcacagcttggatttgcagccagttacttactcggaacctgcattctggtgttcaatcgcatactatgaactaaaccagagggttggagagaccttccatgcgtcacagccctcgctcactgtagacggcttcacagacccatcaaactcggagaggttctgcttaggcttgctctccaacgttaaccgaaatgccactgtagaaatgacaagaagacatataggaaggggagtgcgcttgtattacataggtggggaagtgtttgctgagtgcctaagtgatagtgcaatctttgtgcagagccccaactgtaaccagagatacggctggcaccctgcaacagtgtgtaagatcccaccaggctgtaacctgaagatcttcaacaaccaagaatttgctgctcttctggctcagtctgtcaaccagggttttgaagccgtttatcagctaacccgaatgtgcaccataagaatgagttttgtgaagggctggggagcagaatatcggaggcagacagtaacaagtactccttgctggattgaacttcatctgaatggccctctgcagtggctggacaaagtattaactcagatgggatccccttcagtgcgatgctcaagcatgtcgtaaacccatcaaagactcgctgtaacagctcctccgtcgtagtattcatgtatgatcccgtggactgtttgctatccaaaaattccagagcaaaaacagcacttgaggtctcatcagttaaagcaccttgtggaatctgtttcctatatttgaatattagatgggaaaattagtgtctagaaatgccctccccagcggggaaaaagaagacttaaagacttaatgatgtcttgttgggcataagacagtatcccaaaggttattaataacagtagtagttgtgtacaggtaatgtgtccagacccagtattgcagtactatgctgtttgtatacattcttagtttgcataaatgaggtgtgtgtgctgcttcttggtctaggcaagcctttataaaattacagtatctaatctgttattcccacttctccgttatttttgtgtcttttttaatatataatatatatatatcaagattttcaaattatcatttagaagcagattttccttgtagaaactaatttttctgccttttaccaaaaataaacaaactcttgggggaaaaaaaaaaaaaaaaaaaaaaaaaaaaaaaaaaaaaaaaaaaaaa

SEQ ID NO: 15 - protein, 494 aa, Mus musculus
MSSILPFTPPVVKRLLGWKKSAGGSGGAGGGEQNGQEEKWCEKAVKSLVKKLKKTGRLDELEKAITTQNCNTKCVTIPSTCSEIWGLSTANTVDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMSEITACISQRQVKELDVLIQTEQWPQYP

SEQ ID NO: 16 - DNA, 6256 bp, Homo sapiens
gcggccgccgcctccgccccgcgttcggggccttcccgaccctgcactgctgccgtccgcccgcccggccgctcttctcttcgccgtgggagccgctccgggcgcagggccgcgcgccgagccccgcaggctgcagcgccgcggcccggcccggcgccccggcaacttcgccgagagttgaggcgaagtttgggcgaccgcggcaggccccggccgagctcccctctgcgcccccggcgtcccgtcgagcccagccccgccgggggcgctcctcgccgcccgcgcgccctccccagccatgtcgtccatcctgcctttcactcccccgatcgtgaagcgcctgctgggctggaagaagggcgagcagaacgggcaggaggagaaatggtgcgagaaggcggtcaagagcctggtcaagaaactcaagaagacggggcagctggacgagctggagaaggccatcaccacgcagaacgtcaacaccaagtgcatcaccatccccaggtccctggatggccggttgcaggtgtcccatcggaaggggctccctcatgtcatctactgccgcctgtggcgatggccagacctgcacagccaccacgagctacgggccatggagctgtgtgagttcgccttcaatatgaagaaggacgaggtctgcgtgaatccctaccactaccagagagtagagacaccagttctacctcctgtgttggtgccacgccacacagagatcccggccgagttccccccactggacgactacagccattccatccccgaaaacactaacttccccgcaggcatcgagccccagagcaatattccagagaccccaccccctggctacctgagtgaagatggagaaaccagtgaccaccagatgaaccacagcatggacgcaggttctccaaacctatccccgaatccgatgtccccagcacataataacttggacctgcagccagttacctactgcgagccggccttctggtgctccatctcctactacgagctgaaccagcgcgtcggggagacattccacgcctcgcagccatccatgactgtggatggcttcaccgacccctccaattcggagcgcttctgcctagggctgctctccaatgtcaacaggaatgcagcagtggagctgacacggagacacatcggaagaggcgtgcggctctactacatcggaggggaggtcttcgcagagtgcctcagtgacagcgctatttttgtccagtctcccaactgtaaccagcgctatggctggcacccggccaccgtctgcaagatcccaccaggatgcaacctgaagatcttcaacaaccaggagttcgctgccctcctggcccagtcggtcaaccagggctttgaggctgtctaccagttgacccgaatgtgcaccatccgcatgagcttcgtcaaaggctggggagcggagtacaggagacagactgtgaccagtaccccctgctggattgagctgcacctgaatgggcctttgcagtggcttgacaaggtcctcacccagatgggctccccaagcatccgctgttccagtgtgtcttagagacatcaagtatggtaggggagggcaggcttggggaaaatggccatgcaggaggtggagaaaattggaactctactcaacccattgttgtcaaggaagaagaaatctttctccctcaactgaaggggtgcacccacctgttttctgaaacacacgagcaaacccagaggtggatgttatgaacagctgtgtctgccaaacacatttaccctttggccccactttgaagggcaagaaatggcgtctgctctggtggcttaagtgagcagaacaggtagtattacaccaccggccccctccccccagactctttttttgagtgacagctttctgggatgtcacagtccaaccagaaacacccctctgtctaggactgcagtgtggagttcaccttggaagggcgttctaggtaggaagagcccgcagggccatgcagacctcatgcccagctctctgacgcttgtgacagtgcctcttccagtgaacattcccagcccagccccgccccgccccgccccaccactccagcagaccttgccccttgtgagctggatagacttgggatggggagggagggagttttgtctgtctccctcccctctcagaacatactgattgggaggtgcgtgttcagcagaacctgcacacaggacagcgggaaaaatcgatgagcgccacctctttaaaaactcacttacgtttgtcctttttcactttgaaaagttggaaggatctgctgaggcccagtgcatatgcaatgtatagtgtctattatcacattaatctcaaagagattcgaatgacggtaagtgttctcatgaagcaggaggcccttgtcgtgggatggcatttggtctcaggcagcaccacactgggtgcgtctccagtcatctgtaagagcttgctccagattctgatgcatacggctatattggtttatgtagtcagttgcattcattaaatcaactttatcatatgctcttttaaatgtttggtttatatattttctttaaaaatcctgggctggcacattgactgggaaacctgagtgagacccagcaactgcttctctcccttctctctcctgaggtgaagcttttccaggttttgttgaagagatacctgccagcacttctgcaagctgaaatttacagaagcaaattcaccagaagggaaacatctcaggccaacataggcaaatgaaaagggctattaaaatatttttacacctttgaaaattgcaggcttggtacaaagaggtctgtcatcttccccctgggatataagatgatctagctcccggtagaggatcaccggtgacaactatagcagttgtattgtgtaacaagtactgctcccagcagcaattagggagaaaactagtctaaattatttcaactggaaaaaagaaaaaagagtcctcttcttttcccagccttttgcagaacacagtagacagaactgccaccttcaattggtactttattctttgctgctgtttttgtataaaatgacctatcccacgtttttgcatgaatttatagcaggaaaaatcaagggatttcctatggaagtcctgctttattccaggtgaagggaaggaagtgtatatacttttggcaagtcatacagctcaaatgtgatgagatttctgatgttagagggagatggagaggcttcctgatgcctcatctgcagggtcctgtgcctctgaagttctagccatgaggtttccaggtaggacagctgctccccaagcctcctgaggacacaggaagagacggaaggagcaccttgacagacttgtgtgagtcttctcgaaggagggttgactcagaacccagagacaatacaaaacccctcacttcctctgagagggccaaatgctgtgagtctgaagtatgtgcctggtgtgaaatgatctatggcctgtttcttacacaggaagccccctgaacctcctgtacatgtgttcatgttcccagccagctctgagacccaggaaccaaatattccattttggcttctgctagagcagtcatggttcctctcctaaaagccatgggcagcagtttccgagggcctgcatgatccacctgctgcacgatcctatgagggcttcctgtggcacacagccctctgggtgcttgggaactagcttcaggcacagcctgattctggtgatccagtgatctatggaagtcgtgtcttactccaggtgaagggggaaaaaaaaagcctatactttggcaggttatgaactttgaatgtgatgaaatgacacgtttggctgcatttggatggtgtcttagaaccctcattgctcagacctgaaggctacttctaggagcatgaagtttgagttttgtgtttttccaaaggatacttccttggccctttttctttattgactagaccaccagaggaggatgtgtgggattgtaggcaaacccacctgtggcatcactgaaaataaatttgatcatacctaagaggttaggaaatggtgccattcccaccttagagtgctacataggtgctttgggcgtatgtaacattagtgtccttccttgaagccacaagctagttttcttagttttaaaatcctgttgtatgaatggcatttgtatattaaaacacttttttaaaggacagttgaaaagggcaagaggaaaccagggcagttctagaggagtgctggtgactggatagcagttttaagtggcgttcacctagtcaacacgaccgcgtgtgttgcccctgccctgggctccccgccatgacatcttcaccttgcagcttgtgctgagactgacccaagtgcagctagcactgggacacagatccttgtcttcagcaccttccaaggagccaacttttattccctttcctctctcccctccccacctcgcttcttcccaatttagtaacttagatgcttccagcacatacgtaggtagctaccccagccggtttggattacaggcctgtgctggaacatcatctcagttggccaccttcctggcaggctgtagacctgacattttgagacaagcctagagtcaggagcagggactttgactcttaggaagagcacacatgagggcaaggctgctggcagacgtctccattgtccttatgttgtctgtgttgtatttttttttttttattgaccatggtgattatttttttaaaccatcgttaatatactgaagtgagctatagcacatatcatgtgcttagtttgtttatttttctccatctccccttggcttcctagagtttggacatattccaggctaaatgcttttactcaagactacagaaaggtttgaagtagtgtgtgcatggcatgcacgtatgtaagtaatctggggaagaagcaaagatctgtttcattcttagcctcaggcctcatgagggtctccacagggccggagctcaggttacaccactccttcgtccttacaggagatgtagggagaagaatctgcaggctgcttgtaggactgttcaccaagggggataccagcagcaagagagtgcacccgtttagccctggaccctgtttcttactgtgtgacttggctagagttgggagttcccccaaaataaacgtgtccccattttaccagaaccaaacctcaacacagcgaagctgtactgtctttgtgtggcaaagatgttcccttgtaggcccctttcaggtaaccgtcttcacaatgtattttcatcacagtttaaggagcatcagccgcttctcaagtgggtagggaaagcagaaaaacgtacgcaagaggacatggatccaaaatgatgatgaagcatctcccatggggaggtgatggtggggagatgatgggctaaacaggcaacttttcaaaaacacagctatcatagaaaagaaacttgcctcatgtaaactggattgagaaattctcagtgattctgcaatggatttttttttaatgcagaagtaatgtatactctagtattctggtgtttttatatttatgtaataatttcttaaaaccattcagacagataactatttaattttttttaagaaagttggaaaggtctctcctcccaaggacagtggctggaagagttggggcacagccagttctgaatgttggtggagggtgtagtggctttttggctcagcatccagaaacaccaaaccaggctggctaaacaagtggccgcgtgtaaaaacagacagctctgagtcaaatctgggcccttccacaagggtcctctgaaccaagccccactcccttgctaggggtgaaagcattacagagagatggagccatctatccaagaagccttcactcaccttcactgctgctgttgcaactcggctgttctggactctgatgtgtgtggagggatggggaatagaacattgactgtgttgattaccttcactattcggccagcctgaccttttaataactttgtaaaaagcatgtatgtatttatagtgttttagatttttctaacttttatatcttaaaagcagagcacctgtttaagcattgtacccctattgttaaagatttgtgtcctctcattccctctcttcctcttgtaagtgcccttctaataaacttttcatggaaaagctcctgtgccaggagctcagtctgaaaaaaaaaaaaaaaaaaaaaaaaa

SEQ ID NO: 17 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 18 - DNA, 3221 bp, Homo sapiens
caacaacacggccctggtcgtcgtcgccgctgcggtaacggagcggtttgggtggcggagcctgcgttcgcgccttcccactcccctcgccaccgcccgagcccaggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttgttctgctttatcttttcataaagggttgaaaatgtgtttgctgccttgctcctagcagacagaaactggattaaaacaattttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaagtaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtctcactgttctgcaaaggtggcaatgcttaaactaaataatgaataaactgaatattttggaaactgctaaattctatgttaaatactgtgcagaataatggaaacattacagttcataataggtagtttggatatttttgtacttgatttgatgtgactttttttggtataatgtttaaatcatgtatgttatgatattgtttaaaattcagtttttgtatcttggggcaagactgcaaacttttttatatcttttggttattctaagccctttgccatcaatgatcatatcaattggcagtgactttgtatagagaatttaagtagaaaagttgcagatgtattgactgtaccacagacacaatatgtatgctttttacctagctggtagcataaataaaactgaatctcaacataaaaaaaaaaaaaaaaaaaa

SEQ ID NO: 19 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 20 - DNA, 6504 bp, Mus musculus
gagagcgcgcctgggcggcggggaggacttgcactaagaagaagcctatggcacctgtcaagttaaatgtcactccccgcctccacttggactttctgcttaagacctgcatgtgacttttcacctgcgagccacgcttttggtatctactgactttgattacaggaaagtgtctgaagatttgtatcaaatgacgtcaatggccagcttgttttctttcactagtccagccgtgaagcgattgttgggctggaaacaaggtgacgaggaagagaaatgggcagaaaaggcagtggatgctttagtgaaaaagctgaagaagaagaagggtgctatggaggagctggagaaagccttgagcagcccaggacagccaagcaagtgtgtcacgatccccaggtccttggatggacgtctgcaagtttctcacaggaaaggcttgccccatgttatatattgccgtgtttggcgctggccagatttgcagagccatcacgagctaaaaccattggatatttgtgaatttccttttggatctaagcaaaaggaagtttgtatcaatccataccactataagagagtggagagtccagtcttacctccagtattagtgcctcgtcacaatgaattcaatccacaacacagccttctggttcagttcaggaacctgagccacaatgaaccgcacatgccacaaaacgccacgtttcccgattctttccaccaacccaacaacgctcctttccccttatctcctaacagcccctatcctccttcccctgctagcagcacatatcccaactccccagcaagctctggacctggaagtccatttcaactcccagctgacacccctccccctgcctatatgccacctgatgatcagatggccccagataattcccagcctatggatacaagcagtaacatgattcctcagaccatgcccagcatatccagcagagatgttcagcctgtcgcctatgaggagcccaaacactggtgttcgattgtctactatgaattaaacaatcgtgttggggaagcttttcatgcatcttctactagtgtgttagtagatggatttacagatccttcaaataacaaaagtagattctgcctgggattgttgtcaaatgttaatcgtaattcaactattgaaaacactaggcggcatattggaaaaggtgttcatctatactacgttggtggggaggtgtacgctgagtgtcttagtgacagcagcatctttgttcagagtaggaactgcaactttcaccatggcttccatcccaccaccgtctgtaagatccccagcagctgcagcctcaagatttttaacaatcaggagtttgctcagcttctggctcagtcagtcaaccatggattcgaggctgtgtatgagctcaccaagatgtgtaccattcgaatgagctttgtcaagggctggggagcagagtaccaccgacaggacgtcaccagtactccctgctggattgagattcacctccacgggcctctgcagtggctggataaagtccttactcagatgggctctccgctgaaccccatttcttctgtttcatagtgcagaagtattctttcaactatatttttagtggacttgttttaattttagaggaatttccagtacagatgctgtgagctgacatggaaaacagatattattttttctacgtaattgtgaccaacacatttgtattttatgatgatattacatttgtttgtattcgtgttcattgtgattaactttcaaaagtattgtaaacgatgtagagtattttgcccctgttgaaatgtttagcattgatcttaaactggaacgtactttttcttattgtcccaacgttttttaatttgttaaattttttttacaaagtagttcatcacataatgaaattttatcctataagagaacatatattgtggaaagcagtagatgatatttctctgggaatttctttgccttaccacctttgaaaaagcatacattgtttgcaaaacctcaaagtagggcttgcttaaaggaaactgttgaatcttgtttgaaggacactgcagtcctaacgtgttcagtgaaagcaaggtggtagatttctggacgtcatacatttacatttaatataggtaatattcatcagtgtaatgtgacttcatgccatatatattttgtaaaacaattcctttttaaaaacttcaagtatttctcatttactcaaatttgttgtaagtcctacttaacagttagttactatgtgctctgtggccttgttcagcattgtttgctgctttgggccaacaattcaagaactctaattttcctgtgcattaatcttttcattttgcacttttatgggtgactgtcttagtgtagcctctggtaaaatactattaggtggcctggttttagagctcctcctcgctgccttggcactcctttgtgcaacacgaccacttagagatgacagctgtgagctgtgctgctttttctagcctttaatttccaatgtagtttataatgttgttcttctatagctccagctaaggtgcctgttagtcccctacaatgttatgagcattattgacattgaaaggttatgtatgtatgaatacctttgctccttaccagacttgtcatacaaggactcgtgcagtgtagccagtagaggctctttggttggcccaagaatgaggctgttggtgtaagtgaatcacaatagggattgggatagttcatgtcatatgtcatatagcaagacaatgtagagtgtaggcttgtctctctgcatcaacgctctgcctctttctttttatccttttagaacctacatggacgctaatctccacaacactgttggatgtgaacactcttaagacactcatctagttcactgtgccttgtccttaggactcttaaccactttctagggagcagttatggcctgagatggacagtcatggcctgagaatgaagacactactttgataaagaaaaaggcctcatttgcctatcagagtgagaaaggtttttttctggtgccttttgaaaatatacagagccacttggttcttctgctgaaaatgtaattttggtttgactttttagagtgcccttcctgcctttatgaggaaaacagctatttttttttttggggggggggattccttttgttttctgtgccattatttattgcctttcagagtgcaaccattgggtggctttgctccttcagagagggctccttgatagccttcagtagcttgagctgtagacataagtattccatagcaagagtgtgtcagctccatgagagagatgtctgctttatagccgaggcagaaaccgttcatgttcctttacttggcagccttcaggaacaggtttgtaagaacgtgtcttgagttgagtgagtgtatgtctgtgagctctgctgaagtctggacacaagggccttgcctgctccttttttcagcagtgggttacatgttgtctctccacagtcttcatgtcataggtctcggacttgcagagtcctatgtggcctgccatctgtacagtggcaggactgaagctctgagctgttctgaggttcatggagaaatcccaacctattctgtggtcagtaaatggagactgtgtagtctacctgctcctgtactgtccttactgtatgtaaggatatacagacgcctgtgggtaggcagtactcacagtgagatgaagacagcaagtgtgcactgaaccacagagggcagggagtagggcctctgaagaagccaccagaccagaccagtgccggtacagtctttgtcagagatggctctgatggggcccagactgaccctgaccatgctgagttgctgagggtagccttcagttctctaccctctgaagtgctaggatgacagacatccgccatcatacccagcttccgtggtgctaaggatcagcctcagtcttcaggcgtgctaggcatgtactttgccaagtatttagtatacaaaatacattagtatctgccagggaaaaaagatttgcaaataataaagattgccatcagtttgataaatgttgtaaatggaagaatcaaaatctcagcgatggattacagcaacaagatgctgcctaggaaaagcaggaccaagaggtacatttgactagtataccttcagcgtagcgtgatgacctcactgatgtcacccaactgaacttaagggctgtaagtaggcgtgctgtgggccttccagaactagagaaaattataggaggaagtcagttctaaagtatcaaaagctgggtaatggtggcacatgcctttgattctagcactcgggaagcaggggctagcctagtctacagagcaacttctacacagagaaactgtcttggaggaaaataaaaaaagaaaagtcaaagagcaaacaaatagaacagagtaggaatccgtgtccccttttttctatgtttcacggttgcaggtgtaagaaaagtagtcatagatgtggctgagtttctaagatgaaaccagtagtaagattgctaaatataacacttcaaccaagttaaacaccctttgggggtatgaatgaaagtaacactgcaatatgaaatgaaccgtgcaagtaacacttggggttacctcacagtctccctatgcctgagaggactgtgggaaacatttccatcccctgccagtatcgccattgggaggacagagtagatgaagaagtgaagtcttactggtccagggcacgcctgtcagcaatgccatttgtgcttctgccacagagagcaccgagaggcttggctcagtatcctcgaaccttctctggtcacttccctggcagcacttgggtccctgtcactcactggtctcttaaaagtcccgtctctttgcttcctaaagattctctaaaaaaattactattttttatttcttttttaaaagtctttgttattttgttttgggatacagtctctttgtacagtcctggctggcctggaaattactatgtaggccagcctcaaacttgaagtaattctcttgcctctgcctctggagttctgggattacaggcatgcactgcagagtacagtgagctctgatggcttttaaaattcagcccctttgagggtttggttttagatccattagctttgtctgaacccatctttgtccggccgagtaaatcctctgctatccggggtctcggtagaaatgtgttctcagtatacatacgactaaacattggttgtttataggtagcctcagatatttggtagagcatcttttttgaaagtaatctccagctaggtgggtatttccctcacagcagtaggattttccctttaggagataccagttcttcatctttcttgtgaaaataatgcctttatggggagtgaagattaaggagttgtttctacactaacagaattctatttgatggacaacttggacagttctgtggacttgggtgggttctagtgtgctaagaaggataacagtatttaatagtgtctgtcatcaggccttgctcatctccctgtctagggctgtaggtcagtgctcgagcacttagcaggcatcgagtctagtgttcagtgcccagcattgcacagaactcagaatatatctgtactgaaactgaagtgaccacctacaaccaggtggtatgccagaaccacagaaaggagattcacggtgatgtgtttaaagcattgggctggtgacggttgctgtgtagtaatgacctcttcctcagcaaagagagtcctggagcaggctgtcctcagaagagggaagggactggtgtgctccttgtgcagataacttagtgtataaatcggcatgagtagctatcctttaaggatttgtttgaagttactctttgtaaaaagttgagaattttgtgtgcagttgggcacatgcttgcccttcccccacccgccatagtcctgcctctcttgctgtgaactggtgtcagctacaacactccagctaggtctgagctcttttgagagaaggtctcgtagagcaccattctcagagagaagctaaagcatggggagccttaggacggtcaggcaatgcactctttaccacggctggctaaggctgcagcttgaccgtccttacctaaatcaggtaagaatgtgattacagagcgagtgcttgtgttccccggcctgccttctccgaggaagatgcttcatccgaggatgatgcagagcagacgatggctgcactgtaggtctgcctccttctgtgtatgggttctgctgctgcttacggcataggaaagtacactagcagcgtgcttcaattctgccatcttttgatacttataaaaatgtattaggttttactgtattgtgctctcaaagccataactcttaagaaatttggtttttttgcatattgtttgctaatactttgttttaataaacctcaaaatctgcttaaaaaaaaaaaaaaaaaa

SEQ ID NO: 21 - protein, 465 aa, Mus musculus
MTSMASLFSFTSPAVKRLLGWKQGDEEEKWAEKAVDALVKKLKKKKGAMEELEKALSSPGQPSKCVTIPRSLDGRLQVSHRKGLPHVIYCRVWRWPDLQSHHELKPLDICEFPFGSKQKEVCINPYHYKRVESPVLPPVLVPRHNEFNPQHSLLVQFRNLSHNEPHMPQNATFPDSFHQPNNAPFPLSPNSPYPPSPASSTYPNSPASSGPGSPFQLPADTPPPAYMPPDDQMAPDNSQPMDTSSNMIPQTMPSISSRDVQPVAYEEPKHWCSIVYYELNNRVGEAFHASSTSVLVDGFTDPSNNKSRFCLGLLSNVNRNSTIENTRRHIGKGVHLYYVGGEVYAECLSDSSIFVQSRNCNFHHGFHPTTVCKIPSSCSLKIFNNQEFAQLLAQSVNHGFEAVYELTKMCTIRMSFVKGWGAEYHRQDVTSTPCWIEIHLHGPLQWLDKVLTQMGSPLNPISSVS

SEQ ID NO: 22 - DNA, 2887 bp, Homo sapiens
gaattcggcacgaggagcgatcgagggagctgagccgagagaaagagccgccgggcgctgcctcgccagacctcgctgggaccccggggccaccgggaggcacttttgtggaggggggagggggggcgacctcggcagcctcggcgcacgaagcgtccgagggcagcgtggggygggctgcgacctctgcatcggtggactgcatttttaattaaggattcccagcagctctttgggatttttacagcttccactcatgtgttgacacccgcgtccaggagaaactcgctccaagtgcatctagcgcctgggacctgagacggcgttggcctttcgtgcatgcaaatccagggatttaggttttgtttgggatttccttttctttctttccttttttttttctttttgcagggagtaagaagggagctgggggtatcaacaagcctgcctttcggatcctgcgggaaaagcccatgtagttaagcgctttggtttaaaaaaaaggcaaggtaaaggcagggctttccagacacatttaggggttcgcgcgagctttgtgctcatggaccagccgcacaacttttgaaggctcgccggcccatgtggggtctttctggcgggccgcctgcagcccccctaaagcgcggggggctggagttgttgagcagccccgccgctgtggtccatgtagccgctggccgcgcgcggaatgcggcttcgcgtgcgcgtgttcccggccgtcccgccttggcgaggtccctcatgttgttgccctgcggcgcccctttgaagacaggctgtgcgcggtttgcacggcggtccgcggcggaggttcatgtgggggtgcgacccgcgcagccggcgccttgttgagggaacggacccccggtaaccggagaccgcctcccccccacccctggcgccaaaggatattgtatgttcaggtccaaacgctcggggctggtgcggcgactttggcgaagtcgtgtggtccccaaccgggaggaaggcggcagcggcggcggcggtggcggcgacgaggatgggagcttgggcagccgagctgagccggccccgcgggcaagagagggcggaggctgcggccgctccgaagtccgcccggtagccccgcggcggccccgggacgcagtgggacagcgaggcgcccagggcgcggggaggcgccggcgcgcagggggccccccgaggcccatgtcggagccaggggccggcgctgggagctccctgctggacgtggcggagccgggaggcccgggctggctgcccgagagtgactgcgagacggtgacctgctgtctcttttcggagcgggacgccgccggcgcgccccgggacgccagcgaccccctggccggggcggccctggagccggcgggcggcgggcggagtcgcgaagcgcgctcgcggctgctgctgctggagcaggaactcaaaaccgtcacgtactcgctgctgaagcggctcaaggagcgctcgctggacacgctgctggaggcggtggagtcccgcggcggcgtgccgggcggctgcgtgctggtgccgcgcgccgacctccgcctgggcggccagcccgcgccgccgcagctgctgctcggccgcctctttcgctggcccgacctgcagcacgccgtggagctgaagcccctgtgcggctgccacagcttcgccgccgccgccgacggccctaccgtgtgctgcaacccctaccacttcagccggctctgcgggcccgaatctccgccacctccctactctcggctgtctcctcgcgacgagtacaagccactggatctgtccgattccacattgtcttacactgaaacggaggctaccaactccctcatcactgctccgggtgaattctcagacgccagcatgtctccggacgccaccaagccgagccactggtgcagcgtggcgtactgggagcaccggacgcgcgtgggccgcctctatgcggtgtacgaccaggccgtcagcatcttctacgacctacctcagggcagcggcttctgcctgggccagctcaacctggagcagcgcagcgagtcggtgcggcgaacgcgcagcaagatcggcttcggcatcctgctcagcaaggagcccgacggcgtgtgggcctacaaccgcggcgagcaccccatcttcgtcaactccccgacgctggacgcgcccggcggccgcgccctggtcgtgcgcaaggtgccccccggctactccatcaaggtgttcgacttcgagcgctcgggcctgcagcacgcgcccgagcccgacgccgccgacggcccctacgaccccaacagcgtccgcatcagcttcgccaagggctgggggccctgctactcccggcagttcatcacctcctgcccctgctggctggagatcctcctcaacaaccccagatagtggcggccccggcgggaggggcgggtgggaggccgcggccaccgccacctgccggcctcgagaggggccgatgcccagagacacagcccccacggacaaaaccccccagatatcatctacctagatttaatataaagttttatatattatatggaaatatatattatacttgtaattatggagtcatttttacaatgtaattatttatgtatggtgcaatgtgtgtatatggacaaaacaagaaagacgcactttggcttataattctttcaatacagatatattttctttctcttcctccttcctcttccttactttttatatatatatataaagaaaatgatacagcagagctaggtggaaaagcctgggtttggtgtatggtttttgagatattaatgcccagacaaaaagctaataccagtcactcgataataaagtattcgcattataaaaaaga

SEQ ID NO: 23 - protein, 496 aa, Homo sapiens
MFRSKRSGLVRRLWRSRVVPNREEGGSGGGGGGDEDGSLGSRAEPAPRAREGGGCGRSEVRPVAPRRPRDAVGQRGAQGAGRRRRAGGPPRPMSEPGAGAGSSLLDVAEPGGPGWLPESDCETVTCCLFSERDAAGAPRDASDPLAGAALEPAGGGRSREARSRLLLLEQELKTVTYSLLKRLKERSLDTLLEAVESRGGVPGGCVLVPRADLRLGGQPAPPQLLLGRLFRWPDLQHAVELKPLCGCHSFAAAADGPTVCCNPYHFSRLCGPESPPPPYSRLSPRDEYKPLDLSDSTLSYTETEATNSLITAPGEFSDASMSPDATKPSHWCSVAYWEHRTRVGRLYAVYDQAVSIFYDLPQGSGFCLGQLNLEQRSESVRRTRSKIGFGILLSKEPDGVWAYNRGEHPIFVNSPTLDAPGGRALVVRKVPPGYSIKVFDFERSGLQHAPEPDAADGPYDPNSVRISFAKGWGPCYSRQFITSCPCWLEILLNNPR

SEQ ID NO: 24 - DNA, 3103 bp, Homo sapiens
cggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggtcagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgttttttcctatgggtgttatcacctagctgaatgtttttctaaaggagtttatgttccattaaacgatttttaaaatgtacacttgaaaaaaaaaaaaaaaaa

SEQ ID NO: 25 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 26 - DNA, 1600 bp, Mus musculus
tttgaatcggaccccactgcttccagaaggaaaggcaagcttgttcctatgacatccgtggacaggtacttgccgccgacctgcccggggccctgcaagccttgaaaggtctcatcctctttccccgtgcagcagcctgagctctgcctcctatgcaccccagcacccccatcagctccctcttctccttcaccagccccgcagtgaagcggctgctgggctggaagcagggagatgaagaggagaagtgggcagagaaggcggtggactctttggtgaagaagttaaagaagaagaaaggcgccatggatgaactggagagggcgctgagctgcccgggtcagcctagcaagtgtgtcaccatcccacggtccctcgatggacgcctccaggtgtcccaccgaaaggggctgccccacgtcatctactgccgcgtgtggcgctggccagacctgcagtcccatcatgagctgaagcccttggagtgctgtgagttcccgttcggctccaagcagaaggaggtctgcatcaacccataccattaccgcagagtggagaccccagttctgcctccagtgctggtaccaagacacagcgagtacaaccctcagctcagcctcctggccaagttccgaagtgcctcgctgcacagcgaacccctcatgccgcacaacgccacctaccctgactctttccagcagtctctctgtccggcaccgccctcctcgccaggccatgtgtttccgcagtctccatgccccaccagctacccgcactcccccggaagtccttccgagtcagacagtccctatcaacactcagacttccggccagtttgctacgaggaaccccagcactggtgttctgttgcctactacgaactaaacaaccgggtcggagagactttccaggcgtcctcgcggagcgtgctcatagacggcttcaccgacccttccaataacaggaataggttttgccttgggcttctctcaaatgtaaacagaaactcgaccatagaaaacaccaggaggcacattggaaagggtgtgcatttgtactacgttgggggcgaggtgtatgcggagtgcgtgagcgacagcagcatctttgtccagagccggaactgcaactaccagcacggcttccacccggccaccgtctgcaagatccccagcggctgcagcctcaaggtcttcaacaaccagctcttcgcccagctgctcgcccagtccgtgcaccacggctttgaagtggtgtatgagctgacgaagatgtgcacgattcggatgagctttgtgaagggctggggagcagagtatcatcgccaggatgtcacgagcaccccctgctggatcgagatccatcttcatggaccgctgcagtggttggataaggtgctcactcagatgggctccccacacaaccctatctcttcagtgtcttaagtcacgtcgtcagccacgttgccacagaacagactcgggcaggggcttccatcgtggcaaccgcagctaatgcagggttccggatgcagatgtaaatacacgtgtaacgcatccgagtcacgtttatatcaccgtttgttttgtgctacctactt

SEQ ID NO: 27 - protein, 430 aa, Mus musculus
MHPSTPISSLFSFTSPAVKRLLGWKQGDEEEKWAEKAVDSLVKKLKKKKGAMDELERALSCPGQPSKCVTIPRSLDGRLQVSHRKGLPHVIYCRVWRWPDLQSHHELKPLECCEFPFGSKQKEVCINPYHYRRVETPVLPPVLVPRHSEYNPQLSLLAKFRSASLHSEPLMPHNATYPDSFQQSLCPAPPSSPGHVFPQSPCPTSYPHSPGSPSESDSPYQHSDFRPVCYEEPQHWCSVAYYELNNRVGETFQASSRSVLIDGFTDPSNNRNRFCLGLLSNVNRNSTIENTRRHIGKGVHLYYVGGEVYAECVSDSSIFVQSRNCNYQHGFHPATVCKIPSGCSLKVFNNQLFAQLLAQSVHHGFEVVYELTKMCTIRMSFVKGWGAEYHRQDVTSTPCWIEIHLHGPLQWLDKVLTQMGSPHNPISSVS

(19) United States
(12) Patent Application Publication
(10) Pub. No.: US 2008/0293654 A1
(43) Pub. Date: Nov. 27, 2008

(54) Therapeutic methods using Smads

(76) Inventors: John C. Lee, San Antonio, TX (US); Lee-Chuan C. Yeh, San Antonio, TX (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 11/499,750

(22) Filed: Aug. 3, 2006

Related U.S. Application Data
(63) Continuation of application No. PCT/US05/03229, filed on Feb. 4, 2005.
(60) Provisional application No. 60/542,260, filed on Feb. 4, 2004.

(51) Int. Cl. A61K 31/7088; C12N 5/00; A61P 43/00

(52) U.S. Cl. 514/44; 435/375

(57) ABSTRACT

Methods of inducing the expression of a Smad in a cell or tissue comprising the step of contacting the cell or tissue capable of expressing the Smad with a bone morphogenic protein are provided. Methods of inducing tissue formation and repairing a tissue defect or regenerating tissue, at a target locus in a mammal comprising the step of administering to the target locus a Smad are also provided.

This application is a continuation of International application No. PCT/US2005/003229, filed Feb. 4, 2005, which claims the benefit of U.S. Provisional application No. 60/542,260, filed Feb. 4, 2004, the entire disclosures of which are incorporated by reference herein.

### TECHNICAL FIELD OF THE INVENTION

The present invention relates to methods for tissue formation, repair and regeneration using Smads.

### BACKGROUND OF THE INVENTION

The TGF-β superfamily represents a large number of evolutionarily conserved proteins with diverse activities involved in growth, differentiation, cell migration, development, apoptosis and tissue morphogenesis and repair. This large family includes the bone morphogenic proteins (BMPs), TGF-βs and activins. Each subgroup of proteins initiates a unique signaling cascade activated by the formation of a complex with a receptor. The receptors for this family of proteins are type I also known as activin receptor-like kinases (ALKs) and type II serine/threonine kinases. Several such receptors have been identified thus far. The type II receptors are constitutively active. Upon ligand binding the type II receptor phosphorylates particular serine and threonine residues in the type I receptor. The type I serine/threonine kinases become activated and transduce signals to downstream molecules.

The downstream molecules in the signaling cascade include the Smads. These molecules are vertebrate counterparts to the Drosophila and Caenorhabditis elegans, proteins known as Mad (mothers against dpp) and Sma, respectively. The name Smad originates from a fusion between Mad and Sma. In recent years, several Smads have been identified (Smad 1-8) (Derynck R. et al., 1996, “Nomenclature: Vertebrate mediators of TGF-β family signals”, Cell, 18, 173). The Smads can be divided into three groups: receptor-regulated Smads (R-Smads), common-partner Smads (Co-Smads) and inhibitory Smads (I-Smads). R-Smads are bound to the cell membrane through membrane bound proteins. They transiently interact with and are activated by phosphorylation by activated type I receptor kinases. R-Smads include Smad1, Smad2, Smad3, Smad5 and Smad8. Of those, Smad2 and Smad3 are TGF-β- and activin-specific, whereas Smad1, Smad5 and Smad8 are BMP-specific. The activated R-Smads recruit Co-Smads and these heteromeric complexes translocate to the nucleus. Co-Smads include Smad4. These nuclear Smad complexes bind to DNA directly or indirectly through other DNA-binding proteins, and regulate the transcription of target genes. I-Smads interact with the activated type I receptor, and prevent R-Smads from interacting with activated type I receptors. I-Smads include Smad6 and Smad7.

As described above, the TGF-β superfamily of proteins have important roles in various physiological events including the inductive properties of the proteins belonging to the BMP family. Therefore, there remains a need for identifying means useful for promoting tissue regeneration in patients with traumas caused, for example, by injuries or degenerative disorders.

The ability to induce Smad protein expression in sufficient quantities at a target locus would be very useful in orthopedic medicine, certain types of plastic surgery, dental and various periodontal and craniofacial reconstructive procedures, and procedures generally involving bone, cartilage, tendon, ligament and neural regeneration. Several Smad genes are now cloned and may be recombinantly expressed in a variety of host systems. The ability to recombinantly produce active Smads, including variants and fragments thereof, and to express them at a target locus makes potential therapeutic treatments using these proteins either alone or together with BMPs feasible.

### SUMMARY OF THE INVENTION

This invention is based on the discovery that Smad expression is induced in the presence of various bone morphogenic proteins such as OP-1 (BMP-7) and CDMPs. Therefore, this invention provides a method of inducing the expression of a Smad in a cell or tissue comprising the step of contacting the cell or tissue capable of expressing a Smad with a bone morphogenic protein (BMP). In some embodiments, the tissue is selected from the group consisting of bone, cartilage, tendon, ligament and neural tissue. In one preferred embodiment, the tissue is bone or cartilage. In another preferred embodiment, the tissue is tendon or ligament. In a more preferred embodiment, the tissue is bone. In another more preferred embodiment, the tissue is cartilage.

In some embodiments, the cells used in the methods of this invention are progenitor cells. In some embodiments, the progenitor cells include an osteoprogenitor cell, a cartilage progenitor cell, a ligament progenitor cell, a tendon progenitor cell, or a neural progenitor cell. In a preferred embodiment, the progenitor cell is an osteoprogenitor cell or a cartilage progenitor cell. In another preferred embodiment, the progenitor cell is a tendon progenitor cell or a ligament progenitor cell. In a more preferred embodiment, the progenitor cell is an osteoprogenitor cell. In another more preferred embodiment, the progenitor cell is a cartilage progenitor cell.

In some embodiments, the cell or tissue is contacted with more than one BMP. In some embodiments, the cell or tissue is contacted with two BMPs. BMPs include, but are not limited to, OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, and NEURAL. In one preferred embodiment, the BMP is OP-1 (BMP-7). In another preferred embodiment, the BMP is CDMP-1 or GDF-5. In yet another preferred embodiment, the first bone morphogenic protein is OP-1 and the second bone morphogenic protein is CDMP-1 or GDF-5.

In some embodiments, the Smads used in the methods of the present invention include Smad1, Smad2, Smad3, Smad5 and Smad8. In a preferred embodiment, the Smad is Smad5. In another preferred embodiment, the Smad is a recombinant Smad.

In some embodiments, the cell or tissue used in the method of inducing the expression of a Smad in a cell or tissue is further capable of expressing a serine/threonine kinase receptor. In some embodiments the serine/threonine kinase receptor is selected from the group consisting of type I and type II receptors. In some embodiments, only type I receptors are used. In some embodiments, only type II receptors are used. In some embodiments, both type I and type II receptors are used. Preferably, the type I and type II receptors are recombinant. In some embodiments the serine/threonine kinase receptors are linked to an expression control sequence. In some embodiments, the expression control sequence comprises a constitutive promoter. In some embodiments, the expression control sequence comprises an inducible promoter. In some embodiments, the type I receptor is an activin receptor-like kinase (ALK). ALKs include but are not limited to ALK-1, ALK-2, ALK-3, ALK-4, ALK-5, ALK-6, ALK-7 and fragments thereof.

The invention also provides gene therapy methods of inducing tissue formation, repairing a tissue defect or regenerating tissue at a target locus. In some embodiments, the invention provides a method of inducing tissue formation at a target locus in a mammal comprising the step of administering to the target locus a nucleic acid encoding a Smad. In some embodiments, the invention provides a method of inducing tissue formation at a target locus in a mammal comprising the step of administering to the target locus a vector comprising a nucleic acid encoding a Smad operably linked to an expression control sequence. In some embodiments, the invention provides a method of inducing tissue formation at a target locus in a mammal comprising the step of administering to the target locus a cell comprising a vector comprising a nucleic acid encoding a Smad operably linked to an expression control sequence.

The invention further provides a method of repairing a tissue defect or regenerating tissue at a target locus in a mammal comprising the step of administering to the target locus a nucleic acid encoding a Smad. In some embodiments, the invention provides a method of repairing a tissue defect or regenerating tissue at a target locus in a mammal comprising the step of administering to the target locus a vector comprising a nucleic acid encoding a Smad operably linked to an expression control sequence. In some embodiments, the invention provides a method of repairing a tissue defect or regenerating tissue at a target locus in a mammal comprising the step of administering to the target locus a cell comprising a vector comprising a nucleic acid encoding a Smad operably linked to an expression control sequence.

In some embodiments, the tissue in the gene therapy methods of the present invention is selected from the group consisting of bone, cartilage, tendon, ligament and neural tissue. In one preferred embodiment, the tissue is bone or cartilage. In another preferred embodiment, the tissue is tendon or ligament. In a more preferred embodiment, the tissue is bone. In another more preferred embodiment, the tissue is cartilage.

In some embodiments, the cells used in the gene therapy methods of this invention are progenitor cells. In some embodiments, the progenitor cells include an osteoprogenitor cell, a cartilage progenitor cell, a ligament progenitor cell, a tendon progenitor cell, or a neural progenitor cell. In a preferred embodiment, the progenitor cell is an osteoprogenitor cell or a cartilage progenitor cell. In another preferred embodiment, the progenitor cell is a tendon progenitor cell or a ligament progenitor cell. In a more preferred embodiment, the progenitor cell is an osteoprogenitor cell. In another more preferred embodiment, the progenitor cell is a cartilage progenitor cell.

In some embodiments, the expression control sequence operably linked to the nucleic acid encoding a Smad comprises a constitutive promoter. In other embodiments, the expression control sequence operably linked to the nucleic acid encoding a Smad comprises an inducible promoter. A Smad according to this invention includes, but is not limited to, Smad1, Smad2, Smad3, Smad5, Smad8 and fragments thereof. In a preferred embodiment, the Smad is Smad5. In another preferred embodiment, the Smad is a recombinant Smad.

In some embodiments, the methods of inducing tissue formation, repairing a tissue defect or regeneration tissue of this invention further comprise the step of administering to the target locus a serine/threonine kinase receptor. In some embodiments, a nucleic acid encoding a serine/threonine kinase receptor is administered. In some embodiments, a vector comprising a nucleic acid encoding a serine/threonine kinase receptor operably linked to an expression control sequence is administered. In other embodiments, a cell comprising a vector comprising a nucleic acid encoding a serine/threonine kinase receptor operably linked to an expression control sequence is administered.

In some embodiments, the expression control sequence operably linked to the a serine/threonine kinase receptor comprises a constitutive promoter. In some embodiments, the expression control sequence operably linked to the serine/threonine kinase receptor comprises an inducible promoter.

In some embodiments, the serine/threonine kinase receptor used in the gene therapy methods of inducing tissue formation, repairing a tissue defect or regenerating tissue at a target locus, is selected from the group consisting of type I and type II receptors.

In some embodiments, only type I receptors are used. In some embodiments, only type II receptors are used. In some embodiments both type I and type II receptors are used. Preferably recombinant type I and recombinant type II receptors are used. In some embodiments, the type I receptor is an activin receptor-like kinase (ALK). The ALKs that may be used in the present invention include, but are not limited to ALK-1, ALK-2, ALK-3, ALK-4, ALK-5, ALK-6, ALK-7, and fragments thereof. In a preferred embodiment, the ALK is ALK-2, ALK-3, ALK-6 and fragments thereof.

In some embodiments, the methods of inducing tissue formation, repairing a tissue defect or regeneration tissue of this invention further comprise the step of administering to the target locus a bone morphogenic protein. In some embodiments, the bone morphogenic protein is administered as a nucleic acid. In some embodiments, the bone morphogenic protein is administered as a vector comprising a nucleic acid encoding the bone morphogenic protein operably linked to an expression control sequence. In some embodiments, the bone morphogenic protein is administered as a cell comprising a vector comprising a nucleic acid encoding a bone morphogenic protein operably linked to an expression control sequence. In some embodiments, the expression control sequence linked to the BMP nucleic acid comprises a constitutive promoter. In some embodiments, the expression control sequence linked to the BMP nucleic acid comprises an inducible promoter.

The bone morphogenic protein according to this invention includes, but is not limited to, OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, and fragments thereof. In some embodiments, one BMP is administered. In some embodiments, more than one BMP is administered. In some embodiments, two BMPs are administered. In some embodiments, the first BMP is OP-1 and the second BMP is CDMP-1 or GDF-5. In a preferred embodiment, the BMP is OP-1. In another preferred embodiment, the BMP is CDMP-1 or GDF-5.

### BRIEF DESCRIPTION OF THE DRAWINGS

FIG. 1 is a Western blot analysis for Smad5 protein in C2C12 cells following treatment with OP-1 and CDMP-1. Lane 1 is the Smad5 levels in control C2C12 cells. Lane 2 is the Smad5 levels in CDMP-1 treated (200 ng/ml) C2C12 cells. Lane 3 is the Smad5 levels in OP-1 treated (100 ng/ml) C2C12 cells. Lane 4 is the Smad5 levels in cells treated with both OP-1 (100 ng/ml) and CDMP-1 (200 ng/ml).

FIG. 2 is a plasmid map of pW24 containing the OP-1 gene.

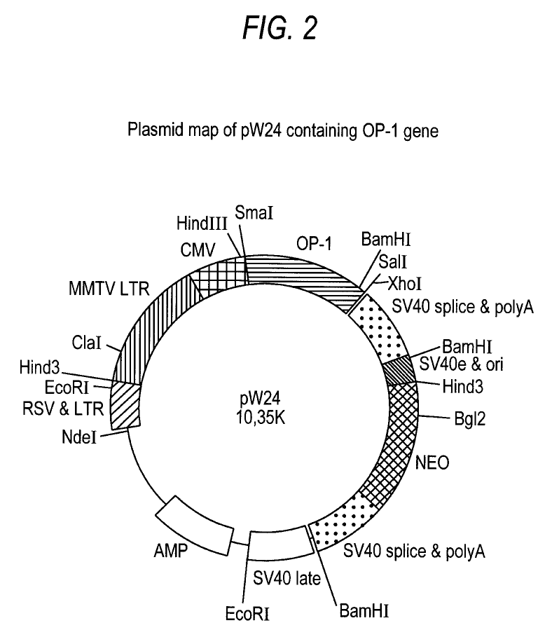

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “defect” or “defect site,” refers to a disruption of the specified tissue. A defect can assume the configuration of a “void”, which is understood to mean a three-dimensional defect such as, for example, a gap, cavity, hole or other substantial disruption in the structural integrity of the tissue (e.g., bone, chondral, osteochondral, neural, ligament, tendon). Moreover, a defect can also be a detachment of the tendon or ligament from its point of attachment to bone, cartilage or muscle. In certain embodiments, the defect is such that it is incapable of endogenous or spontaneous repair. A defect can be the result of accident, disease, and/or surgical manipulation.

The term “target locus” refers to the site (e.g., a defect) in any tissue that is in need of repair or regeneration. The target locus need not be a defect sit. It may be any site where bone, cartilage, tendon, ligament or neural tissue regeneration is desired.

The term “repair” refers to new tissue formation which is sufficient to at least partially fill the void or structural discontinuity at the defect site. Repair does not, however, mean, or otherwise necessitate, a process of complete healing or a treatment, which is 100% effective at restoring a defect to its pre-defect physiological/structural/mechanical state.

The term “therapeutically effective amount” refers to an amount effective to repair, regenerate, promote, accelerate, prevent degradation, or form tissue.

The term “patient” refers to an animal, including a mammal (e.g., a human).

The term “bone morphogenic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain, which characterizes the BMP protein family. Preferably, the protein has at least 70% amino acid sequence identity with at least one known BMP family member within the conserved C-terminal cysteine rich domain. Members of the BMP family may have less than 50% DNA or amino acid sequence identity overall. Bone morphogenic proteins may be monomeric, homo- or hetero-dimeric. Bone morphogenic proteins include osteogenic proteins.

Bone morphogenic proteins are capable of inducing progenitor cells to proliferate and/or to initiate differentiation pathways that lead to cartilage, bone, tendon, ligament or other types of tissue formation depending on local environmental cues, and thus bone morphogenic proteins may behave differently in different surroundings. For example, a bone morphogenic protein may induce bone tissue at one treatment site and cartilage tissue at a different treatment site. Bone morphogenic proteins include full length proteins as well as fragments thereof.

The term “osteogenic protein (OP)” refers to a bone morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include but are not limited to, osteogenically active forms of OP-1, OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL, conservative amino acid sequence variants thereof having osteogenic activity and fragments thereof. In one currently preferred embodiment, osteogenic protein includes any one of: OP-1, CDMP-1, CDMP-2, CDMP-3, GDF-5, GDF-6, GDF-7, amino acid sequence variants and homologs thereof, including species homologs thereof and fragments thereof. Particularly preferred osteogenic proteins are those comprising an amino acid sequence having at least 70% homology with the C-terminal 102-106 amino acids, defining the conserved seven cysteine domain, of, e.g., human OP-1. Certain preferred embodiments of the instant invention comprise the osteogenic protein, OP-1. As further described elsewhere herein, the osteogenic proteins suitable for use with this invention can be identified by means of routine experimentation using the art-recognized bioassay described by Reddi and Sampath (Sampath et al., Proc. Natl. Acad. Sci., 84, pp. 7109-13, incorporated herein by reference).

Proteins useful in this invention include eukaryotic proteins identified as osteogenic proteins (see U.S. Pat. No. 5,011,691, incorporated herein by reference), such as the OP-1, OP-2, OP-3 and CBMP-2 proteins, as well as amino acid sequence-related proteins, such as DPP (from Drosophila), Vg1 (from Xenopus), Vgr-1 (from mouse), GDF-1 (from humans, see Lee, PNAS, 88, pp. 4250-4254 (1991)), 60A (from Drosophila, see Wharton et al., PNAS, 88, pp. 9214-9218 (1991)), dorsalin-1 (from chick, see Basler et al., Cell, 73, pp. 687-702 (1993) and GenBank accession number L12032) and GDF-5 (from mouse, see Storm et al., Nature, 368, pp. 639-643 (1994)). The teachings of the above references are incorporated herein by reference. BMP-3 is also preferred. Additional useful proteins include biosynthetic morphogenic constructs disclosed in U.S. Pat. No. 5,011,691, incorporated herein by reference, e.g., COP-1, COP-3, COP-4, COP-5, COP-7 and COP-16, as well as other proteins known in the art. Still other proteins include osteogenically active forms of BMP-3b (see Takao, et al., Biochem. Biophys. Res. Comm., 219, pp. 656-662 (1996)). BMP-9 (see WO 95/33830), BMP-15 (see WO 96/35710), BMP-12 (see WO 95/16035), CDMP-1 (see WO 94/12814), CDMP-2 (see WO 94/12814), BMP-10 (see WO 94/26893), GDF-1 (see WO 92/00382), GDF-10 (see WO95/10539), GDF-3 (see WO 94/15965) and GDF-7 (see WO95/01802). The teachings of the above references are incorporated herein by reference. BMPs (identified by sequence homology) must have demonstrable osteogenic activity in a functional bioassay to be osteogenic proteins according to this invention.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred bone morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pair wise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “fragment thereof” or “fragment” refers to a stretch of at least about 5 amino acid residues. In some embodiments, this term refers to a stretch of at least about 10 amino acid residues. In other embodiments, it refers to a stretch of at least about 15 to 20 amino acid residues. The fragments may be naturally derived or synthetically generated. To be active, any fragment must have sufficient length to display biological activity.

### Methods Using Smads

The present invention provides a method of inducing the expression of a Smad in a cell or tissue comprising the step of contacting the cell or tissue capable of expressing the Smad with a bone morphogenic protein.

This invention also provides gene therapy methods for inducing tissue formation at a target locus, repairing a tissue defect or regenerating tissue at a target locus using Smads. In some embodiments, the methods comprise the step of administering to the target locus a nucleic acid encoding a Smad. In some embodiments, the methods comprise the step of administering to the target locus a vector comprising a nucleic acid encoding a Smad operably linked to an expression control sequence. In some embodiments, the methods comprise the step of administering a cell comprising a vector comprising a nucleic acid encoding a Smad operably linked to an expression control sequence. In some embodiments, the expression control sequence operably linked to a Smad nucleic acid comprises an inducible promoter. In some embodiments, the expression control sequence operably linked to a Smad nucleic acid comprises a constitutive promoter.

A Smad according to the present invention is a R-Smad. R-Smads includes, but are not limited to, Smad1, Smad2, Smad3, Smad5, Smad8 or fragments thereof. In a preferred embodiment, the Smad is Smad5. In another embodiment the Smad is recombinant.

In some embodiments, the gene therapy methods further comprise the step of administering to the target locus a pharmaceutically effective amount of a BMP. In some embodiments, the methods further comprise the step of administering a nucleic acid encoding a BMP. In some embodiments, the methods further comprise the step of administering a vector comprising a nucleic acid encoding a BMP operably linked to an expression control sequence. In some embodiments, the methods further comprise the step of administering a cell comprising a vector comprising a nucleic acid encoding a BMP operably linked to an expression control sequence. In some embodiments, the expression control sequence operably linked to a BMP nucleic acid comprises an inducible promoter. In some embodiments, the expression control sequence operably linked to a BMP nucleic acid comprises a constitutive promoter.

The BMPs according to the invention include but are not limited to, OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, and NEURAL. (see infra, for discussion of BMPs). In a preferred embodiment, the BMP is OP-1 (BMP-7). In another preferred embodiment, the BMP is CDMP-1 or GDF-5.

In some embodiments, more than one BMP is administered. In one preferred embodiment, two BMPs are administered. In yet another preferred embodiment, the first BMP is OP-1 and the second BMP is CDMP-1 or GDF-5. In another embodiment three BMPs are administered. In some embodiments, the BMP is recombinant.

In some embodiments, the gene therapy methods of this invention further comprise the step of administering a serine/threonine kinase receptor. In some embodiments, the methods of this invention further comprise the step of administering to the target locus a nucleic acid encoding a serine/threonine kinase receptor. In some embodiments, the methods of this invention further comprise the step of administering to the target locus a vector comprising a nucleic acid encoding a serine/threonine kinase receptor operably linked to an expression control sequence. In some embodiments, the methods of this invention further comprise the step of administering to the target locus a cell comprising a vector comprising a nucleic acid encoding a serine/threonine kinase receptor operably linked to an expression control sequence. In some embodiments, the serine/threonine kinase receptor is selected from the group consisting of type I and type II receptors. In some embodiments, only a type I receptor is used. In some embodiments, only a type II receptors is used. In some embodiments both type I and type II receptors are used. Preferably the type I and type II receptors are recombinant. In some embodiments, the expression control sequence operably linked to a serine/threonine kinase receptor nucleic acid comprises an inducible promoter. In some embodiments, the expression control sequence operably linked to a serine/threonine kinase receptor nucleic acid comprises a constitutive promoter.

In some embodiments, the type I receptor is an activin receptor-like kinase (ALK). The ALKs according to this invention include, but are not limited to ALK-1, ALK-2, ALK-3, ALK-4, ALK-5, ALK-6, ALK-7, and fragments thereof. Preferred ALKs are ALK-2, ALK-3 and ALK-6.

### Bone Morphogenic Protein (BMP) Family

The BMP family, named for its representative bone morphogenic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are bone morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The Drosophila DPP and Xenopus Vg-1 gene products are 50% identical to each other (and 35-40% identical to TGF-β). Both the Dpp and Vg-1 products are morphogenic proteins that participate in early patterning events during embryogenesis of their respective hosts. These products appear to be most closely related to mammalian bone morphogenetic proteins BMP-2 and BMP-4, whose C-terminal domains are 75% identical with that of Dpp.

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

The naturally occurring bone morphogenic proteins share substantial amino acid sequence homology in their C-terminal regions (domains). Typically, the above-mentioned naturally occurring osteogenic proteins are translated as a precursor, having an N-terminal signal peptide sequence typically less than about 30 residues, followed by a “pro” domain that is cleaved to yield the mature C-terminal domain of approximately 100-140 amino acids. The signal peptide is cleaved rapidly upon translation, at a cleavage site that can be predicted in a given sequence using the method of Von Heijne, Nucleic Acids Research, 14, pp. 4683-4691 (1986). The pro domain typically is about three times larger than the fully processed mature C-terminal domain.

Another characteristic of the BMP protein family members is their ability to dimerize. Several bone-derived osteogenic proteins (OPs) and BMPs are found as homo- and heterodimers in their active forms. The ability of OPs and BMPs to form heterodimers may confer additional or altered morphogenic inductive capabilities on bone morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In one preferred embodiment of this invention, the BMPs independently comprise a pair of subunits disulfide bonded to produce a dimeric species, wherein at least one of the subunits comprises a recombinant peptide belonging to the BMP protein family. In another preferred embodiment of this invention, the BMPs independently comprise a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation. In some embodiments, the BMPs are each independently monomers.

In preferred embodiments, the pair of morphogenic polypeptides have amino acid sequences each comprising a sequence that shares a defined relationship with an amino acid sequence of a reference morphogen. Herein, preferred osteogenic polypeptides share a defined relationship with a sequence present in osteogenically active human OP-1, SEQ ID NO: 1. However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein.

As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins, (see U.S. Pat. No. 5,011,691 and Oppermann et al., incorporated herein by reference), as well as the proteins referred to as BMP-2, BMP-3, BMP-4 (see WO 88/00205, U.S. Pat. No. 5,013,649 and WO 91/18098, incorporated herein by reference), BMP-5 and BMP-6 (see WO 90/11366, PCT/US90/01630, incorporated herein by reference), BMP-8 and BMP-9.

Preferred BMPs of this invention comprise at least one polypeptide selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, NEURAL and amino acid sequence variants and homologs thereof, including species homologs thereof and fragments thereof. In some embodiments, one BMP is used. In some embodiments, more than one BMP is used. In some embodiments, two BMPs are used. In some embodiments, three BMPs are used. In some embodiments, the first BMP is OP-1 (BMP-7) or a fragment thereof, and the second BMP is selected from the group consisting of CDMP-1, CDMP-2, CDMP3, GDF-5, GDF-6, GDF-7 and fragments thereof. In some embodiments, the second BMP is CDMP-1, GDF-5 or a fragment thereof. In some embodiments, the first BMP is OP-1 and the second BMP is CDMP-2 or GDF-5.

Publications disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO 94/10203 (PCT US93/10520)), BMP-2, BMP-3, BMP-4, (WO 88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO 94/26893 (PCT/US94/05290); BMP-11 (WO 94/26892 (PCT/US94/05288); BMP-12 (WO95/16035 (PCT/US94/14030); BMP-13 (WO95/16035 (PCT/US94/14030); GDF-1 (WO 92/00382 (PCT/US91/04096) and Lee et al. PNAS, 88, pp. 4250-4254 (1991); GDF-8 (WO 94/21681 (PCT/US94/03019); GDF-9 (WO 94/15966 (PCT/US94/00685); GDF-10 (WO 95/10539 (PCT/US94/11440); GDF-11 (WO 96/01845 (PCT/US95/08543); BMP-15 (WO 96/36710 (PCT/US96/06540); MP-121 (WO 96/01316 (PCT/EP95/02552); GDF-5 (CDMP-1, MP52) (WO 94/15949 (PCT/US94/00657) and WO 96/14335 (PCT/US94/12814) and WO 93/16099 (PCT/EP93/00350)); GDF-6 (CDMP-2, BMP13) (WO 95/01801 (PCT/US94/07762) and WO 96/14335 and WO 95/10635 (PCT/US94/14030)); GDF-7 (CDMP-3, BMP12) (WO 95/10802 (PCT/US94/07799) and WO 95/10635 (PCT/US94/14030)). The above publications are incorporated herein by reference.

In another embodiment of this invention, the BMPs may be prepared synthetically. BMPs prepared synthetically may be native, or may be non-native proteins, i.e., those not otherwise found in nature. Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes. Purified fusion proteins may be cleaved, refolded, implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3). Oppermann et al., U.S. Pat. Nos. 5,011,691 and 5,324,819, which are incorporated herein by reference, describe the amino acid sequences of COP-5 and COP-7 as shown below:

| | |
|---|---|
| COP5 | LYVDFS-DVGWDDWIVAPPGYQAFYCHGECPFPLAD |
| COP7 | LYVDFS-DVGWNDWIVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (−) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family.

In certain preferred embodiments, the BMPs useful herein independently include those in which the amino acid sequences comprise a sequence sharing at least 70% amino acid sequence homology or “similarity”, preferably 80%, more preferably 90%, even more preferably 95%, even more preferably 98% homology or similarity, with a reference bone morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In some embodiments, the BMP comprises a dimeric protein having an amino acid sequence having at least 70% homology within the C-terminal 102-106 amino acids of human OP-1. In certain embodiments, a polypeptide suspected of being functionally equivalent to a reference BMP polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. In one preferred embodiment, the reference sequence is OP-1. In another preferred embodiment, the reference sequence is selected from CDMP-1, CDMP-2 or CDMP-3. Bone morphogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred bone morphogenic polypeptides share at least 60% amino acid identity with the preferred reference sequence of human OP-1, still more preferably at least 65% amino acid identity therewith.

In another embodiment, useful BMPs include those sharing the conserved seven cysteine domain and sharing at least 70% amino acid sequence homology (similarity) within the C-terminal active domain, as defined herein. In still another embodiment, the BMPS of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6) are 97 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-5, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8, respectively), as well as alternative residues for the variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

```
Generic Sequence 7
                        Leu Xaa Xaa Xaa Phe Xaa Xaa
                        1 5
Xaa Gly Trp Xaa Xaa Xaa Xaa Xaa Xaa Pro
                10 15
Xaa Xaa Xaa Xaa Ala Xaa Tyr Cys Xaa Gly
                20 25
Xaa Cys Xaa Xaa Pro Xaa Xaa Xaa Xaa Xaa
                30 35
Xaa Xaa Xaa Asn His Ala Xaa Xaa Xaa Xaa
                40 45
Xaa Xaa Xaa Xaa Xaa Xaa Xaa Xaa Xaa Xaa
                50 55
Xaa Xaa Xaa Cys Cys Xaa Pro Xaa Xaa Xaa
                60 65
Xaa Xaa Xaa Xaa Xaa Leu Xaa Xaa Xaa Xaa
                70 75
Xaa Xaa Xaa Val Xaa Leu Xaa Xaa Xaa Xaa
                80 85
Xaa Met Xaa Val Xaa Xaa Cys Xaa Cys Xaa
                90 95
```

wherein each Xaa independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res. 11=(Gln, Leu, Asp, His, Asn or Ser); Xaa at res.12=(Asp, Arg, Asn or Glu); Xaa at res.13=(Trp or Ser); Xaa at res.14=(Ile or Val); Xaa at res.15=(Ile or Val); Xaa at res.16 (Ala or Ser); Xaa at res.18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

SEQ ID NO: 9
Cys Xaa Xaa Xaa Xaa
1 5

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res.7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res.5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP-10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 97 amino acid sequence that accommodates the C-terminal six cysteine skeleton and, like Generic Sequence 8, Generic Sequence 10 is a 102 amino acid sequence which accommodates the seven cysteine skeleton.

```
Generic Sequence 9
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “res.” means “residue” and Xaa at res.1=(Phe, Leu or Glu); Xaa at res.2=(Tyr, Phe, His, Arg, Thr, Lys, Gln, Val or Glu); Xaa at res.3=(Val, Ile, Leu or Asp); Xaa at res.4=(Ser, Asp, Glu, Asn or Phe); Xaa at res.5=(Phe or Glu); Xaa at res.6=(Arg, Gln, Lys, Ser, Glu, Ala or Asn); Xaa at res.7=(Asp, Glu, Leu, Ala or Gln); Xaa at res.8=(Leu, Val, Met, Ile or Phe); Xaa at res.9=(Gly, His or Lys); Xaa at res.10=(Trp or Met); Xaa at res.11=(Gln, Leu, His, Glu, Asn, Asp, Ser or Gly); Xaa at res.12=(Asp, Asn, Ser, Lys, Arg, Glu or His); Xaa at res.13=(Trp or Ser); Xaa at res.14=(Ile or Val); Xaa at res.15=(Ile or Val); Xaa at res.16=(Ala, Ser, Tyr or Trp); Xaa at res. 18=(Glu, Lys, Gln, Met, Pro, Leu, Arg, His or Lys); Xaa at res.19=(Gly, Glu, Asp, Lys, Ser, Gln, Arg or Phe); Xaa at res.20=(Tyr or Phe); Xaa at res.21=(Ala, Ser, Gly, Met, Gln, His, Glu, Asp, Leu, Asn, Lys or Thr); Xaa at res.22=(Ala or Pro); Xaa at res.23=(Tyr, Phe, Asn, Ala or Arg); Xaa at res.24=(Tyr, His, Glu, Phe or Arg); Xaa at res.26=(Glu, Asp, Ala, Ser, Tyr, His, Lys, Arg, Gln or Gly); Xaa at res.28=(Glu, Asp, Leu, Val, Lys, Gly, Thr, Ala or Gln); Xaa at res.30=(Ala, Ser, Ile, Asn, Pro, Glu, Asp, Phe, Gln or Leu); Xaa at res.31=(Phe, Tyr, Leu, Asn, Gly or Arg); Xaa at res.32=(Pro, Ser, Ala or Val); Xaa at res.33=(Leu, Met, Glu, Phe or Val); Xaa at res.34=(Asn, Asp, Thr, Gly, Ala, Arg, Leu or Pro); Xaa at res.35=(Ser, Ala, Glu, Asp, Thr, Leu, Lys, Gln or His); Xaa at res.36=(Tyr, His, Cys, Ile, Arg, Asp, Asn, Lys, Ser, Glu or Gly); Xaa at res.37=(Met, Leu, Phe, Val, Gly or Tyr); Xaa at res.38=(Asn, Glu, Thr, Pro, Lys, His, Gly, Met, Val or Arg); Xaa at res.39=(Ala, Ser, Gly, Pro or Phe); Xaa at res.40=(Thr, Ser, Leu, Pro, His or Met); Xaa at res.41=(Asn, Lys, Val, Thr or Gln); Xaa at res.42=(His, Tyr or Lys); Xaa at res.43=(Ala, Thr, Leu or Tyr); Xaa at res.44=(Ile, Thr, Val, Phe, Tyr, Met or Pro); Xaa at res.45=(Val, Leu, Met, Ile or His); Xaa at res.46=(Gln, Arg or Thr); Xaa at res.47=(Thr, Ser, Ala, Asn or His); Xaa at res.48=(Leu, Asn or Ile); Xaa at res.49=(Val, Met, Leu, Pro or Ile); Xaa at res.50=(His, Asn, Arg, Lys, Tyr or Gln); Xaa at res.51=(Phe, Leu, Ser, Asn, Met, Ala, Arg, Glu, Gly or Gln); Xaa at res.52=(Ile, Met, Leu, Val, Lys, Gln, Ala or Tyr); Xaa at res.53=(Asn, Phe, Lys, Glu, Asp, Ala, Gln, Gly, Leu or Val); Xaa at res.54=(Pro, Asn, Ser, Val or Asp); Xaa at res.55=(Glu, Asp, Asn, Lys, Arg, Ser, Gly, Thr, Gln, Pro or His); Xaa at res.56=(Thr, His, Tyr, Ala, Ile, Lys, Asp, Ser, Gly or Arg); Xaa at res.57=(Val, Ile, Thr, Ala, Leu or Ser); Xaa at res.58=(Pro, Gly, Ser, Asp or Ala); Xaa at res.59=(Lys, Leu, Pro, Ala, Ser, Glu, Arg or Gly); Xaa at res.60=(Pro, Ala, Val, Thr or Ser); Xaa at res.61=(Cys, Val or Ser); Xaa at res.63=(Ala, Val or Thr); Xaa at res.65=(Thr, Ala, Glu, Val, Gly, Asp or Tyr); Xaa at res.66=(Gln, Lys, Glu, Arg or Val); Xaa at res.67=(Leu, Met, Thr or Tyr); Xaa at res.68=(Asn, Ser, Gly, Thr, Asp, Glu, Lys or Val); Xaa at res.69=(Ala, Pro, Gly or Ser); Xaa at res.70=(Ile, Thr, Leu or Val); Xaa at res.71=(Ser, Pro, Ala, Thr, Asn or Gly); Xaa at res.72=(Val, Ile, Leu or Met); Xaa at res.74=(Tyr, Phe, Arg, Thr, Tyr or Met); Xaa at res.75=(Phe, Tyr, His, Leu, Ile, Lys, Gln or Val); Xaa at res.76=(Asp, Leu, Asn or Glu); Xaa at res.77=(Asp, Ser, Arg, Asn, Glu, Ala, Lys, Gly or Pro); Xaa at res.78=(Ser, Asn, Asp, Tyr, Ala, Gly, Gln, Met, Glu, Asn or Lys); Xaa at res.79=(Ser, Asn, Glu, Asp, Val, Lys, Gly, Gln or Arg); Xaa at res.80=(Asn, Lys, Thr, Pro, Val, Ile, Arg, Ser or Gln); Xaa at res.81=(Val, Ile, Thr or Ala); Xaa at res.82=(Ile, Asn, Val, Leu, Tyr, Asp or Ala); Xaa at res.83=(Leu, Tyr, Lys or Ile); Xaa at res.84=(Lys, Arg, Asn, Tyr, Phe, Thr, Glu or Gly); Xaa at res.85=(Lys, Arg, His, Gln, Asn, Glu or Val); Xaa at res.86=(Tyr, His, Glu or Ile); Xaa at res.87=(Arg, Glu, Gln, Pro or Lys); Xaa at res.88=(Asn, Asp, Ala, Glu, Gly or Lys); Xaa at res.89=(Met or Ala); Xaa at res.90=(Val, Ile, Ala, Thr, Ser or Lys); Xaa at res.91=(Val or Ala); Xaa at res.92=(Arg, Lys, Gln, Asp, Glu, Val, Ala, Ser or Thr); Xaa at res.93=(Ala, Ser, Glu, Gly, Arg or Thr); Xaa at res.95=(Gly, Ala or Thr); Xaa at res.97=(His, Arg, Gly, Leu or Ser). Further, after res.53 in rBMP3b and mGDF-10 there is an Ile; after res.54 in GDF-1 there is a T; after res.54 in BMP3 there is a V; after res.78 in BMP-8 and Dorsalin there is a G; after res.37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

SEQ ID NO: 9
Cys Xaa Xaa Xaa Xaa
1 5

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res.1=(Tyr, Phe, His, Arg, Thr, Lys, Gln, Val or Glu)” in Generic Sequence 9 refers to Xaa at res.6 in Generic Sequence 10. In Generic Sequence 10, Xaa at res.2=(Lys, Arg, Gln, Ser, His, Glu, Ala, or Cys); Xaa at res.3=(Lys, Arg, Met, Lys, Thr, Leu, Tyr, or Ala); Xaa at res.4=(His, Gln, Arg, Lys, Thr, Leu, Val, Pro, or Tyr); and Xaa at res.5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful BMPs include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
SEQ ID NO: 4
Cys Xaa Xaa His Glu Leu Tyr Val Ser Phe Xaa Asp
1 5 10
Leu Gly Trp Xaa Asp Trp Xaa Ile Ala Pro Xaa Gly
                15 20
Tyr Xaa Ala Tyr Tyr Cys Glu Gly Glu Cys Xaa Phe
25 30 35
Pro Leu Xaa Ser Xaa Met Asn Ala Thr Asn His Ala
                        40 45
Ile Xaa Gln Xaa Leu Val His Xaa Xaa Xaa Pro Xaa
        50 55 60
Xaa Val Pro Lys Xaa Cys Cys Ala Pro Thr Xaa Leu
                                65 70
Xaa Ala Xaa Ser Val Leu Tyr Xaa Asp Xaa Ser Xaa
                75 80
Asn Val Ile Leu Xaa Lys Xaa Arg Asn Met Val Val
85 90 95
Xaa Ala Cys Gly Cys His
                        100
```

wherein Xaa at res.2=(Lys or Arg); Xaa at res.3=(Lys or Arg); Xaa at res.11=(Arg or Gln); Xaa at res.16=(Gln or Leu); Xaa at res.19=(Ile or Val); Xaa at res.23=(Glu or Gln); Xaa at res.26=(Ala or Ser); Xaa at res.35=(Ala or Ser); Xaa at res.39=(Asn or Asp); Xaa at res.41=(Tyr or Cys); Xaa at res.50=(Val or Leu); Xaa at res.52=(Ser or Thr); Xaa at res.56=(Phe or Leu); Xaa at res.57=(Ile or Met); Xaa at res.58=(Asn or Lys); Xaa at res.60=(Glu, Asp or Asn); Xaa at res.61=(Thr, Ala or Val); Xaa at res.65=(Pro or Ala); Xaa at res.71=(Gln or Lys); Xaa at res.73=(Asn or Ser); Xaa at res.75=(Ile or Thr); Xaa at res.80=(Phe or Tyr); Xaa at res.82=(Asp or Ser); Xaa at res.84=(Ser or Asn); Xaa at res.89=(Lys or Arg); Xaa at res.91=(Tyr or His); and Xaa at res.97=(Arg or Lys).

In still another preferred embodiment, useful BMPs have polypeptide chains with amino acid sequences comprising a sequence encoded by a nucleic acid that hybridizes, under low, medium or high stringency hybridization conditions, to DNA or RNA encoding reference BMP sequences, e.g., C-terminal sequences defining the conserved seven cysteine domains of OP-1, OP-2, BMP-2, BMP-4, BMP-5, BMP-6, 60A, GDF-5, GDF-6, GDF-7 and the like. As used herein, high stringent hybridization conditions are defined as hybridization according to known techniques in 40% formamide, 5×SSPE, 5×Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. In some embodiments, the pair of polypeptides are not disulfide bonded. In some embodiments the pair of polypeptides are disulfide bonded. Such disulfide bonded BMPs are inactive when reduced, but are active as oxidized homodimers and when oxidized in combination with others of this invention to produce heterodimers. Thus, members of a folded pair of bone morphogenic polypeptides in a morphogenically active protein can be selected independently from any of the specific polypeptides mentioned above.

The BMPs useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The BMPs contemplated herein can be expressed from intact or truncated cDNA or from synthetic DNAs in prokaryotic or eukaryotic host cells, and purified, cleaved, refolded, and dimerized to form morphogenically active compositions. Alternatively, cells expressing recombinant may be used in the methods of this invention. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins.

### Serine/Threonine Kinase Receptors

TGF-β superfamily members elicit their cellular responses through formation of heteromeric complexes of specific type I and type II serine/threonine kinase receptors. To date five type II receptors and seven type I receptors (also termed activin receptor-like kinases (ALKs) have been identified (See e.g., Derynck et al., Biochem. Biophys. Acta, 1333, pp. F105-F150 (1997); Massague, Annu. Rev. Biochem., 67, pp. 753-791 (1998)). The type II receptors are kinases and are constitutively active. Upon ligand-mediated heteromeric complex formation, the type II receptor phosphorylates particular serine and threonine residues in the type I receptor juxtamembrane region, thereby activating the type I receptor.

ALK-1 was identified as an endothelial specific TGF-β type I receptor (see e.g., Oh et al., Proc. Natl. Acad. Sci. U.S.A., 97, pp. 2626-2631 (2000); see also, Lux et al., J. Biol. Chem., 274, 9984-9992 (1999); U.S. Pat. No. 5,968,752). ALK-2 was identified as a type I receptor for activin, TGF-β and BMPs (see, e.g., Attisano et al., Cell, 75, pp. 671-680 (1993); Miettinen et al., J. Cell Biol., 127, pp. 2021-2036 (1994); ten Dijke et al., Science 264, 101-104 (1994); Macias-Silva et al., J. Cell Biol., 273, pp. 25628-25636 (1998); U.S. Pat. No. 6,271,365). ALK-4 and ALK-5 were identified as activin and TGF-β type I receptors and ALK-3 and ALK-6 were identified as BMP type I receptors (see, e.g., Oh et al., supra and Lux et al., supra; U.S. Pat. Nos. 6,271,365 and 6,207,814). ALK-7 was identified as the type I receptor for Nodal (see, e.g., Reissman et al., Genes Dev., 15, pp. 2010-22 (2001); U.S. Pat. No. 5,891,638).

In some embodiments of the invention, type II receptors are used. In some embodiments of the invention, type I receptors are used. In yet other embodiments of the invention, both type I and type II receptors are used. In some embodiments, the type I receptor is selected from the group consisting of ALK-1, ALK-2, ALK-3, ALK-4, ALK-5, ALK-6, ALK-7, amino acid sequence variants and homologs thereof, including species homologs thereof and fragments thereof. In a preferred embodiment, the type I receptor is ALK-2, ALK-3 or ALK-6. In some embodiments, the type I and type II receptors are recombinant.

Publications disclosing these sequences as well as their chemical and physical properties include: ALK-1 (U.S. Pat. No. 5,968,752); ALK-2, ALK-4 and ALK-5 (U.S. Pat. No. 6,271,365); ALK-3 and ALK-6 (U.S. Pat. No. 6,207,814); ALK-7 (U.S. Pat. Nos. 5,891,638, 5,614,609 and 5,789,565). These publications are incorporated herein by reference.

The type I and type II receptors contemplated herein can be expressed from intact or truncated cDNA or from synthetic DNAs in prokaryotic or eukaryotic host cells (see discussion of protein expression, infra).

Thus, in view of this disclosure and the knowledge available in the art, skilled genetic engineers can express these receptors in various types of host cells, including both prokaryotes and eukaryotes.

### Smads

The Smad family of proteins can be divided into three subfamilies: R-Smads, Co-Smads and I-Smads. The subfamily of R-Smads can be divided into two groups: BMP-Smads and TGF-β/activin-Smads. Smad1, Smad5, and Smad8 are phosphorylated by ALK-1, ALK-2, ALK-3 and ALK-6, and Smad2 and Smad3 are activated by ALK-5 and ALK-4, respectively. Smad2 and Smad3 are also activated by ALK-7 (see e.g., Derynck et al., Biochim. Biophys. Acta, 1333, pp. F105-F150 (1997), Massague, Annu. Rev. Biochem., 67, 753-792 (1998); Itoh et al., Eur. J. Biochem., 267, 6954-6967 (2000)). The phosphorylated R-Smads form heteromeric complexes with Co-Smads. To date only one Co-Smad has been identified: Smad4. I-Smads, i.e., Smad6 and Smad7 inhibit TGF-β family signaling by preventing the activation of R-Smads and Co-Smads (see, e.g., Itoh et al., supra).

In some embodiments, the Smads used in the methods of this invention are selected from the group consisting of Smad1, Smad2, Smad3, Smad5, Smad8, amino acid sequence variants and homologs thereof, including species homologs thereof and fragments thereof. In a preferred embodiment, the Smad is Smad5. In another embodiments, the Smad is recombinant.

The sequences for Smads are included in the following publications or are readily available to the skilled worker from GenBank: Smad1 (rat: GenBank Accession No. AF067727; SEQ ID NO: 12 and 13); Smad2 (mouse: GenBank Accession No. NM_{—}010754; SEQ ID NO: 14 and 15), Smad3 (human: GenBank Accession No. NM_{—}005902; SEQ ID NO: 16 and 17), Smad4 (mouse: Genbank Accession No. NM_{—}008540; human: GenBank Accession No. NM_{—}005359; SEQ ID NO: 18 and 19), Smad5 (rat: GenBank Accession No. NM_{—}021692; SEQ ID NO: 20 and 21), Smad6 (U.S. Pat. Nos. 6,534,476 and 6,270,994; human: GenBank Accession No. NM_{—}005585; SEQ ID NO: 22 and 23), Smad7 (U.S. Pat. Nos. 6,020,464 and 6,251,628; human: GenBank Accession No. NM_{—}005904; SEQ ID NO: 24 and 25), and Smad8 (rat: GenBank Accession No. AF012347; SEQ ID NO: 26 and 27). These publications and GenBank Accession numbers are incorporated herein by reference.

The Smads contemplated herein can be expressed from intact or truncated cDNA or from synthetic DNAs in prokaryotic or eukaryotic host cells (see discussion of protein expression, infra).

Thus, in view of this disclosure and the knowledge available in the art, skilled genetic engineers can express these receptors in various types of host cells, including both prokaryotes and eukaryotes (e.g., progenitor cells).

### Production or Expression of BMPs, Serine/Threonine Kinase Receptors and Smads

The BMPs, serine/threonine kinase receptors and Smads according to this invention may be derived from a variety of sources. They may be isolated from natural sources, or may be produced by expressing an appropriate recombinant DNA molecule in a host cell. In addition, the BMPs, serine/threonine kinase receptors and Smads of this invention may be derived synthetically and synthetic proteins may optionally be expressed from a recombinant DNA molecule in a host cell.

1. Naturally-Derived Proteins

In one embodiment of this invention, the BMPs, serine/threonine kinase receptors and Smads are isolated from natural sources. BMPs, serine/threonine kinase receptors and Smads may be purified from tissue sources, preferably mammalian tissue sources, using conventional physical and chemical separation techniques well known to those of skill in the art.

2. Recombinantly-Expressed Proteins

In another embodiment of this invention, the Smads, BMPs and serine/threonine kinase receptors and are produced by the expression of an appropriate recombinant DNA molecule in a host cell. The DNA and amino acid sequences of Smads, BMPs and serine/threonine kinase receptors have been reported, and methods for their recombinant production are published and otherwise known to those of skill in the art. For a general discussion of cloning and recombinant DNA technology, see Ausubel et al., supra; see also Watson et al., Recombinant DNA, 2d ed. 1992 (W.H. Freeman and Co., New York).

For cloning and expressing Smads, BMPS and serine/threonine kinase receptors, standard recombinant DNA techniques may be used. With the DNA sequence available, a DNA fragment encoding any of these proteins be inserted into an expression vector selected to work in conjunction with a desired host expression system. The DNA fragment is cloned into the vector with the proper transcription control elements. In some embodiments, the expression of the desired protein may be constitutive. In some embodiments, the expression of the desired protein is under the control of an inducible promoter.

Vectors

In some embodiments, the invention provides vectors comprising the nucleic acids encoding Smads, serine/threonine kinase receptors and/or BMPs. The choice of vector and expression control sequences to which the nucleic acids of this invention are operably linked depends on the functional properties desired, e.g., protein expression, and the host cell to be transformed.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status (e.g. concentration of growth factors or BMPs), or a change in temperature, in the host cell medium.

An appropriate vector is selected according to the host system selected. Useful vectors include but are not limited to plasmids, cosmids, bacteriophage, insect and animal viral vectors, including retroviruses, and other single and double-stranded DNA viruses.

In some embodiments, it may be preferable to recombinantly produce a mammalian protein for therapeutic uses in mammalian cell culture systems in order to produce a protein whose structure resembles more closely that of the natural material. Recombinant protein production in mammalian cells requires the establishment of appropriate cells and cell lines that are easy to transfect, are capable of stably maintaining foreign DNA with an unrearranged sequence, and which have the necessary cellular components for efficient transcription, translation, post-translational modification and secretion of the protein. In addition, a suitable vector carrying the gene of interest is necessary.

DNA vector design for transfection into mammalian cells should include appropriate sequences to promote expression of the gene of interest, including: appropriate transcription initiation, termination and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion.

Preferred DNA vectors also include a marker gene and means for amplifying the copy number of the gene of interest. DNA vectors may also comprise stabilizing sequences (e.g., ori- or ARS-like sequences and telomere-like sequences), or may alternatively be designed to favor directed or non-directed integration into the host cell genome.

Substantial progress in the development of mammalian cell expression systems has been made in the last decade and many aspects of the system are well characterized. A detailed review of the production of foreign proteins in mammalian cells, including useful cells, protein expression-promoting sequences, marker genes, and gene amplification methods, is disclosed in M. M. Bendig, Genetic Engineering, 7, pp. 91-127 (1988).

Particular details of the transfection, expression and purification of recombinant proteins are well documented and are understood by those of skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989).

Briefly, among the best characterized transcription promoters useful for expressing a foreign gene in a particular mammalian cell are the SV40 early promoter, the adenovirus major late promoter (AdMLP), the mouse metallothionein-I promoter (mMT-I), the Rous sarcoma virus (RSV) long terminal repeat (LTR), the mouse mammary tumor virus long terminal repeat (MMTV-LTR), and the human cytomegalovirus major intermediate-early promoter (hCMV). The DNA sequences for all of these promoters are known in the art and are available commercially.

One method of gene amplification in mammalian cell systems is the use of the selectable dihydrofolate reductase (DHFR) gene in a dhfr− cell line. Generally, the DHFR gene is provided on the vector carrying the gene of interest, and addition of increasing concentrations of the cytotoxic drug methotrexate (MTX) leads to amplification of the DHFR gene copy number, as well as that of the physically-associated gene of interest. DHFR as a selectable, amplifiable marker gene in transfected chinese hamster ovary cell lines (CHO cells) is particularly well characterized in the art. Other useful amplifiable marker genes include the adenosine deaminase (ADA) and glutamine synthetase (GS) genes.

In one expression system, gene amplification is further enhanced by modifying marker gene expression regulatory sequences (e.g., enhancer, promoter, and transcription or translation initiation sequences) to reduce the levels of marker protein produced. Lowering the level of DHFR transcription increases the DHFR gene copy number (and the physically-associated gene) to enable the transfected cell to adapt to growth in even low levels of methotrexate (e.g., 0.1 μM MTX). As will be appreciated by those skilled in the art, other useful weak promoters, different from those disclosed and preferred herein, can be constructed using standard vector construction methodologies. In addition, other, different regulatory sequences also can be modified to achieve the same effect.

Another gene amplification scheme relies on the temperature sensitivity (ts) of BSC40-tsA58 cells transfected with an SV40 vector. Temperature reduction to 33° C. stabilizes the temperature sensitive SV40 T antigen, which leads to the excision and amplification of the integrated transfected vector DNA thereby amplifying the physically associated gene of interest.

Eukaryotic cell expression vectors are known in the art and are commercially available. Typically, such vectors contain convenient restriction sites for insertion of the desired DNA segment.

Eukaryotic cell expression vectors may include a selectable marker, e.g., a drug resistance gene. The neomycin phosphotransferase (neo) gene (Southern et al., 1982, J. Mol. Anal. Genet. 1:327-341) is an example of such a gene.

To express the desired proteins of this invention, DNAs encoding the proteins (Smads, serine/threonine kinase receptors and/or BMPs are inserted into expression vectors such as plasmids, retroviruses, cosmids, YACs, EBV-derived episomes, and the like. The expression vector and expression control sequences are chosen to be compatible with the expression host cell used. In some embodiments, Smad, serine/threonine kinase receptors and BMP nucleic acids can be inserted into separate vectors. In some embodiments, the Smad, serine/threonine kinase receptor and BMP nucleic acids are inserted into the same expression vector. In some embodiments the Smad and serine/threonine kinase receptor nucleic acids are inserted into the same vector. In some embodiments, the Smad and BMP nucleic acids are inserted into the same vector. Alternatively, any combination of Smad, BMP and/or serine/threonine kinase receptor are inserted into the same vector.

A convenient vector is one that encodes a functionally complete protein. To the extent secretion of a desired protein is required, the recombinant expression vector can also encode a signal peptide that facilitates secretion of the desired protein from a host cell.

Nucleic acid molecules encoding Smads, serine/threonine kinase receptors and/or BMPs, and vectors comprising these nucleic acid molecules, can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., 1972, Proc. Natl. Acad. Sci. USA 69:2110-2114). For transformation of vertebrate cells, electroporation, cationic lipid or salt treatment methods can be employed. See, e.g., Graham et al., 1973, Virology 52:456-467; Wigler et al., 1979, Proc. Natl. Acad. Sci. USA 76:1373-1376f.

Host Cells

Host cells can be prokaryotic or eukaryotic. Useful host cells include but are not limited to bacteria such as E. coli, yeasts such as Saccharomyces and Picia, insect-baculovirus cell system, and primary, transformed or immortalized eukaryotic cells in culture. Preferred eukaryotic host cells include, but are not limited to, yeast and mammalian cells, e.g., Chinese hamster ovary (CHO) cell, NIH Swiss mouse embryo cells NIH-3T3, baby hamster kidney cells (BHK), C2C12 cells and BSC cells. Other useful eukaryotic cells include osteoprogenitor cells, cartilage progenitor cells, tendon progenitor cells, ligament progenitor cells and neural progenitor cells.

The methodology disclosed herein includes the use of COS cells for the rapid evaluation of vector construction and gene expression, and the use of established cell lines for long term protein production.

The choice of cells/cell lines is also important and depends on the needs of the skilled practitioner. Monkey kidney cells (COS) provide high levels of transient gene expression providing a useful means for rapidly testing vector construction and the expression of cloned genes. COS cells are transfected with a simian virus 40 (SV40) vector carrying the gene of interest. The transfected COS cells eventually die, thus preventing the long term production of the desired protein product. However, transient expression does not require the time consuming process required for the development of stable cell lines.

CHO cells are capable of successfully expressing a wide variety of proteins from a broad range of cell types. Thus, while the glycosylation pattern on a recombinant protein produced in a mammalian cell expression system may not be identical to the natural protein, the differences in oligosaccharide side chains are often not essential for biological activity of the expressed protein.

The DHFR gene also may be used as part of a gene amplification scheme for CHO cells. Another gene amplification scheme relies on the temperature sensitivity (ts) of BSC40-tsA58 cells transfected with an SV40 vector. Temperature reduction to 33° C. stabilizes the ts SV40 T antigen which leads to the excision and amplification of the integrated transfected vector DNA, thereby also amplifying the associated gene of interest.

Stable cell lines were established for CHO cells as well as BSC40-tsA58 cells (hereinafter referred to as “BSC cells”). The various cells, cell lines and DNA sequences chosen for mammalian cell expression of the Smads, serine/threonine kinase receptors and BMPs of this invention are well characterized in the art and are readily available. Other promoters, selectable markers, gene amplification methods and cells also may be used to express the Smads, serine/threonine kinase receptors and BMPs of this invention. Particular details of the transfection, expression, and purification of recombinant proteins are well documented in the art and are understood by those having ordinary skill in the art. Further details on the various technical aspects of each of the steps used in recombinant production of foreign genes in mammalian cell expression systems can be found in a number of texts and laboratory manuals in the art. See, e.g., F. M. Ausubel et al., ed., Current Protocols in Molecular Biology, John Wiley & Sons, New York (1989).

### Progenitor Cells

The progenitor cells that are induced to proliferate and/or differentiate in the present invention are preferably mammalian cells. Preferred progenitor cells include mammalian chondroblasts, osteoblasts and neuroblasts, all earlier developmental precursors thereof, and all cells that develop therefrom (e.g., chondroblasts, pre-chondroblasts and chondrocytes). However, any non-mammalian progenitor cells are also likely to be useful in the methods of the present invention. It is, thus, envisioned that when schemes become available for implanting xenogeneic cells into humans without causing adverse immunological reactions, non-mammalian progenitor cells will be useful for tissue regeneration and repair in humans.

In some embodiments, the progenitor cells comprise a nucleic acid encoding a Smad and optionally a nucleic acid encoding one or more serine/threonine kinase receptor and/or a nucleic acid encoding one or more BMP. In some embodiments, the progenitor cells comprise vectors comprising a nucleic acid encoding a Smad and optionally a nucleic acid encoding one or more serine/threonine kinase receptor and/or a nucleic acid encoding one or more BMP. In some embodiments, the nucleic acid encoding Smad, serine/threonine kinase receptor and/or BMP are in one vector. In some embodiments, the nucleic acid encoding Smad, serine/threonine kinase receptor and/or BMP are in different vectors. In some embodiments, the nucleic acids are recombinant.

In some embodiments, only type I serine/threonine kinase receptors are used. In some embodiments, only type II serine/threonine kinase receptors are used. In other embodiments, both type I and type II serine/threonine kinase receptors are used. In some embodiments, more than one BMP will be administered to the desired cell or tissue. In some embodiments, two BMPS will be used. In some embodiments, three BMPs will be used. The particular choice of combination of BMPs and the relative concentrations at which they are combined may be varied systematically to optimize the tissue type induced at a selected treatment site using the procedures described herein. The relative concentrations of the BMPs either alone or in combination that will optimally induce tissue formation when administered to a mammal may be determined empirically by the skilled practitioner using the procedures described herein.

Gene Therapy

The Smad, serine/threonine kinase receptor and/or BMP proteins can be produced in vivo in a mammal, e.g., a human patient, using a gene therapy approach for inducing tissue formation, repairing a tissue defect or regenerating tissue at a target locus. This involves administration of a suitable Smad, serine/threonine kinase receptor and/or BMP protein-encoding nucleic acid operably linked to suitable expression control sequences. Preferably, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. A preferred adenoviral vector has a deletion in its E1 gene or E3 gene. When an adenoviral vector is used, preferably the mammal is not exposed to a nucleic acid encoding a selectable marker gene.

### Formulations

The proteins (e.g., BMPs) of the present invention can be formulated as part of a pharmaceutical composition. The pharmaceutical compositions provided by this invention comprise at least one protein. The compositions of this invention will be administered at an effective dose to induce the particular type of tissue at the treatment site selected according to the particular clinical condition addressed. Determination of a preferred pharmaceutical formulation and a therapeutically effective dose regimen for a given application is well within the skill of the art taking into consideration, for example, the mode of administration, the condition and weight of the patient, the extent of the desired treatment and the tolerance of the patient for the treatment.

Administration of the proteins of this invention, may be accomplished using any of the conventional modes of administration.

The pharmaceutical compositions comprising a protein of this invention may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include oral, parenteral, subcutaneous, intravenous, intralesional or topical administration. In most cases, the pharmaceutical compositions of this invention will be administered in the vicinity of the treatment site in need of tissue regeneration or repair.

The pharmaceutical compositions comprising a protein of this invention may, for example, be placed into sterile, isotonic formulations with or without cofactors which stimulate uptake or stability. The formulation is preferably liquid, or may be lyophilized powder. For example, the BMP of this invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions also will preferably include conventional pharmaceutically acceptable carriers well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered as a dose regimen that depends on the particular tissue treatment.

The pharmaceutical compositions of this invention may also be administered in conjunction with a morphogenic device using, for example, microspheres, liposomes, other microparticulate delivery systems or sustained release formulations placed in, near, or otherwise in communication with affected tissues or the bloodstream bathing those tissues.

Liposomes containing a protein of this invention can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of BMP release.

The proteins of this invention may also be attached to liposomes containing other biologically active molecules such as immunosuppressive agents, cytokines, etc., to modulate the rate and characteristics of tissue induction. Attachment of BMPs to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

The following are examples which illustrate the methods of this invention. These examples should not be construed as limiting: the examples are included for purposes of illustration and the present invention is limited only by the claims.

### EXAMPLE 1

### Effect of CDMP-1 and OP-1 on Smad5 Levels

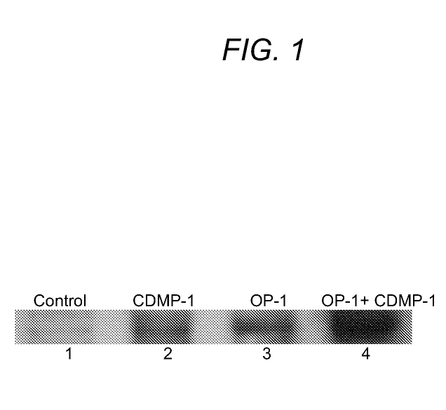

Total C2C12 cell lysates treated with solvent control (lane 1), CDMP-1 (200 ng/ml), or OP-1 (100 ng/ml) were collected after 5 days. Proteins in the lysates were resolved by SDS-PAGE, transferred to nylon membranes, and analyzed using the anti-human Smad5 antibody (Cell Signaling Technology, Inc., Beverly, Mass.), as the primary antibody and anti-rabbit IgG HRP-conjugated antibody was used as the secondary antibody. Immunoreactive bands were detected using the SuperSignal chemiluminescent detection system (Pierce, Rockford, Ill.), according to the manufacturer's instructions. Representative results of two independent experiments are shown. Western blot analyses showed that the Smad5 protein level in control C2C12 cells was hardly detectable by Western blot analysis (FIG. 1, lane 1). CDMP-1 alone stimulated Smad5 protein expression in C2C12 cells (FIG. 1, lane 2). OP-1 alone stimulated Smad5 protein expression to a higher level (FIG. 1, lane 3). The combination of CDMP-1 and OP-1 significantly stimulated Smad5 protein expression even more (FIG. 1, lane 4). The current observation that the protein level of Smad5 was dramatically increased in cells treated with the combination of OP-1 and CDMP-1 would support the supposition that the effect of the combination of OP-1 and CDMP-1 is likely directed at the Smad signaling pathway.

### EXAMPLE 2

### Co-transfection with the OP-1 Gene and the CDMP-1 Gene

Target cells will be transfected with plasmids or viral carriers containing the OP-1 gene and the CDMP-1 gene under an appropriate promoter. In addition, these cells will be transfected with plasmids or viral carriers containing the Smad5 gene. For example, plasmid pW24 (10.35 kb) that contains the coding sequence for OP-1 under the control of the CMV promoter may be used. Similarly, plasmids containing the CMDP-1 gene (pCDMP-1) or the Smad5 gene (pSmad5) will be constructed by replacing the OP-1 gene in the pW24 plasmid. Alternatively, these genes may be placed under the control of promoters that can be induced thus allowing control of expression at will. Additional vectors, such as adenoviral vectors may be constructed and tested in order to optimize and maximize expression.

Target cells that have been shown capable to become osteoblastic upon treatment with OP-1 include osteoblastic cells such as FRC cells, and pluripotent mesenchymal cells such as C2C12 and C3H10T1/2 cell lines. Cells of other orthopaedic utility may also be used. These include cells derived from cartilage, tendon, ligament, and meniscus.

Transfection studies will be carried out using the calcium phosphate-DNA coprecipitation method or FuGene6 (Roche Diagnostics). Briefly, cells will be transfected with the specified plasmid DNAs (1 μg/ml) in serum-free medium for 4-6 h. The medium will be replaced with complete αMEM containing 10% FBS.

### EXAMPLE 3

### Effect of OP-1, CDMP-1 and/or Smad5 on Alkaline Phosphatase Activity

The effect of co-transfection of FRC cells with pW24 and pCDMP-1 and/or pSmad5 will be examined. For example, FRC cells will be transfected with pW24, pCDMP-1 and/or pSmad5. After 48 h, total AP activity will be measured. It is anticipated that the AP activity in cells co-transfected with pW24, pCDMP-1 and/or pSmad5 will increase as a function of pCDMP-1 and/or pSmad5 concentration. The increase should be beyond that in cells transfected with pW24 alone. The AP activity in cells transfected with pCDMP-1 alone will also show a lesser but significant increase compared to the non-transfected control or cells transfected with the empty plasmids (vectors without the OP-1, CDMP-1 and/or Smad5 genes). Co-transfection with pW24 and the empty plasmid is not expected to result in an increase in AP activity beyond that by pW24 alone. Protein levels of OP-1, CDMP-1, and Smad5 under these conditions will be measured by Western blot analysis. Control experiments will include co-transfection of FRC cells with the empty plasmids, i.e. vector without the OP-1, CDMP-1 or Smad5 genes. It is expected that the AP activity will not be elevated beyond the non-transfected controls. The expression of additional osteoblastic cell markers, such as osteocalcin (OC) and bone sialoprotein (BSP) will also be monitored using either Northern blot analysis or real-time PCR. Transfection conditions for other cell types will be optimized accordingly using art recognized methods.

### EXAMPLE 4

### Effect of OP-1, CDMP-1 and/or Smad5 on Mineralized Bone Nodule Formation

The effect of co-transfection of FRC cells with pW24 and pCDMP-1 or pSmad5 on mineralized bone nodule formation will be examined. Confluent FRC cells will be co-transfected with pW24 and pCDM-1 and/or pSmad5 using FuGene6 using the optimal ratio of pW24 to pCDMP-1 or pSmad5 as determined by the experiments described above. Cells will be cultured in complete (MEM containing 5% FBS, ascorbic acid, and β-glycerol phosphate in the presence of 250 μg/ml of Neomycin. Media will be changed every 3 days. Progress of nodule formation will be monitored periodically and the images will be captured using an Olympus CK2 inverted microscope equipped with a CCD camera. At the termination of the experiments, cultures will be stained by Alizarin Red-S staining to assess the extent of formation of mineralized bone nodules.

### EXAMPLE 5

### In Vivo Expression of OP-1, CDMP-1 and Smad5

In vivo studies will be conducted using two experimental approaches: (i) Direct injection of OP-1 expressing vectors together with CDMP-1 and/or Smad5-expressing vectors into muscles of mice, and (ii) injection of transfected cells into muscles.

For direct injection experiments, nude mice will be injected with vectors expressing OP-1, CDMP-1 and/or Smad5 (pW24, pCDMP-1 and/or pSmad5, respectively) with a 27-gauge needle subcutaneously into a male homozygous nude mouse. Standard aseptic techniques will be used in all manipulations. To determine in vivo osteogenic dose response of the vectors, eight mice will be used. Each mouse will be injected with 0.1-10 mg/ml vectors in 100 μl each. Body weight and growth at the site of injection will be followed daily via in-life measurement of the mass. The cross-sectional area of the mass will be measured with a vernier caliper. The size of the mass will be calculated using the formula: length/2×width/2×π. The mass and the body weight will be plotted as a function of time following injection. The animals will be monitored for 49 days. At necropsy, the mass at the site of injection will be collected, fixed, stained with hematoxylin and eosin, and subjected to histological analysis. Controls will include mice injected with individual pW24, pCDMP-1 and/or pSmad5 alone. It is anticipated that the bone mass in mice injected with the combination of the pW24 and pCDMP-1, or pW24 plus pSmad5 will be greater than that injected with individual vector alone.

For experiments using injection of cells, similar experiments as described above will be conducted except that animals will be injected with cells co-transfected with vectors carrying the OP-1, CDMP-1, and/or Smad5 genes. Accordingly, cells will be grown to mid-log phase and transfected with a combination of vectors expressing OP-1, CDMP-1 and/or Smad5 as described above using the optimal ratio of the two vectors. Cells will be removed from the culturing dishes by trypsin-EDTA digestion. Trypsin will be inactivated by serum (10%) and removed by repeated washings with HBSS. Cells will be suspended in a minimal volume of HBSS and injected with a 27-gauge needle subcutaneously into the flank of a male homozygous nude mouse. Standard aseptic techniques will be used in all manipulations. Eight nude mice will be injected with 10⁶ cells in 100 μl each. Outcome measurements as described above will be conducted. It is anticipated that the bone mass in mice injected with cells transfected with the combination of the pW24 and pCDMP-1, or pW24 plus pSmad5 will be greater than that injected with cells transfected with individual vector alone.

### EXAMPLE 6

### Gene Therapy in Patients Using Transfected Cells

For cell therapeutics with transfected genes, appropriate cells will be transfected in vitro with DNA vectors carrying the OP-1 gene, the CDMP-1 gene, and/or the Smad5 gene and optionally a serine/threonine kinase receptor. Appropriate cells include osteoblasts or osteoblastic cell progenitors for the repair of bone defects. For repair of cartilage regeneration, cells of chondrocyte origin or chondrogenitor cells will be appropriate. Similarly, for the regeneration of tendons or ligaments, the appropriate cells include progenitor cells of tendon or ligament origin. The transfected cells will be cultured to allow expression of the transfected gene(s). The cells will then be injected or implanted into a defect site in a patient. The defect site may be in bone, cartilage, tendon, ligament or neural tissue. The number of cells injected or implanted into the defect will depend on the size of the defect. Exemplary DNA vectors will be pW24, pCDMP-1, or pSmad5 as described previously.

### EXAMPLE 7

### Gene Therapy in Patients Using Transfected Cells

For directed gene therapy, a combination of vectors as described above carrying the OP-1 gene, the cDMP-1 gene, the Smad5 gene and/or a serine/threonine kinase will be injected into the defect site in a patient. The genes encoding each of the proteins may be placed in the same vector or in separate vectors.

### EXAMPLE 8

### Monitoring Effects of Gene Therapy in Patients

The repair site will be monitored radiographically every two weeks for a minimum of two years. It is anticipated that the defect site which receives the combination of OP-1+CDMP-1+Smad or OP-1+Smad5 (delivered by either of the methods described in Examples 6 and 7) will exhibit a faster rate of repair than that receives OP-1 alone.

In case of cartilage and tendon/ligament repair, delivery of pCDMP-1 alone to a defect site will induce tissue formation. However, delivery of OP-1+CDMP-1 to a defect site should exhibit a faster rate of repair than delivery of CDMP-1 or Smad5 alone. In all cases, the treated sites should exhibit regeneration whereas those treated with vehicle should not.

1. A method of inducing the expression of a Smad in a cell or tissue comprising contacting the cell or tissue capable of expressing the Smad with a bone morphogenic protein.

2. The method according to claim 1, wherein the cell or tissue is contacted with two bone morphogenic proteins.

3. The method according to claim 1, wherein each bone morphogenic protein is independently selected from the group consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, and NEURAL.

4. The method according to claim 1, wherein the bone morphogenic protein is OP-1.

5. The method according to claim 1, wherein the bone morphogenic protein is CDMP-1 or GDF-5.

6. The method according to claim 2, wherein the first bone morphogenic protein is OP-1 and the second bone morphogenic protein is selected from the group consisting of CDMP-1, and GDF-5.

7. The method according to claim 1, wherein the Smad is selected from the group consisting of Smad1, Smad2, Smad3, Smad5 and Smad8.

8. The method according to claim 7, wherein the Smad is Smad5.

9. The method according to claim 1, wherein the Smad is a recombinant Smad.

10. The method according to claim 1, wherein the cell or tissue is further capable of expressing a serine/threonine kinase receptor.

11. The method according to claim 10, wherein the serine/threonine kinase receptor is a type I receptor.

12. The method according to claim 10, wherein the serine/threonine kinase receptor is a type II receptor.

13. The method according to claim 10, wherein the cell or tissue is further capable of expressing both type I and type II serine/threonine kinase receptor.

14. The method according to claim 11 or 13, wherein the type I receptor is selected from the group consisting of ALK-1, ALK-2, ALK-3, ALK-4, ALK-5, ALK-6, and ALK-7.

15. The method according to claim 10 wherein the serine/threonine kinase receptor is a recombinant serine/threonine kinase receptor.

16. The method according to claim 1 wherein the tissue is selected from the group consisting of bone, cartilage, tendon, ligament and neural tissue.

17. The method according to claim 1 wherein the cell is a progenitor cell.

18. The method according to claim 17, wherein the progenitor cell is selected from the group consisting of an osteoprogenitor cell, a cartilage progenitor cell, a ligament progenitor cell, a tendon progenitor cell, and a neural progenitor cell.

19. A method of inducing tissue formation at a target locus in a mammal comprising the step of administering to the target locus a nucleic acid encoding a Smad.

20. A method of inducing tissue formation at a target locus in a mammal comprising the step of administering to the target locus a vector comprising a nucleic acid encoding a Smad operably linked to an expression control sequence.

21. A method of inducing tissue formation at a target locus in a mammal comprising the step of administering to the target locus a cell comprising a vector comprising a nucleic acid encoding a Smad operably linked to an expression control sequence.

22. A method of repairing a tissue defect or regenerating tissue at a target locus in a mammal comprising the step of administering to the target locus a nucleic acid encoding a Smad.

23. A method of repairing a tissue defect or regenerating tissue at a target locus in a mammal comprising the step of administering to the target locus a vector comprising a nucleic acid encoding a Smad operably linked to an expression control sequence.

24. A method of repairing a tissue defect or regenerating tissue at a target locus in a mammal comprising the step of administering to the target locus a cell comprising a vector comprising a nucleic acid encoding a Smad operably linked to an expression control sequence.

25. The method according to any one of claims 20, 21, 23 or 24, wherein the expression control sequence comprises a constitutive promoter.

26. The method according to claim 20, 21, 23 or 24, wherein the expression control sequence comprises an inducible promoter.

27. The method according to any one of claims 19-24, wherein the Smad is selected from the group consisting of Smad1, Smad2, Smad3, Smad5 and Smad8.

28. The method according to claim 27, wherein the Smad is Smad5.

29. The method according to any one of claims 19-24, wherein the Smad is recombinant Smad.

30. The method according to any one of claims 19-24 further comprising the step of administering to the target locus a nucleic acid encoding a serine/threonine kinase receptor.

31. The method according to any one of claims 19-24 further comprising the step of administering to the target locus a vector comprising a nucleic acid encoding a serine/threonine kinase receptor operably linked to an expression control sequence.

32. The method according to any one of claims 19-24 further comprising the step of administering to the target locus a cell comprising a vector comprising a nucleic acid encoding a serine/threonine kinase receptor operably linked to an expression control sequence.

33. The method according to claim 31, wherein the expression control sequence operably linked to the serine/threonine kinase receptor comprises a constitutive promoter.

34. The method according to claim 31, wherein the expression control sequence operably linked to the serine/threonine kinase receptor comprises an inducible promoter.

35. The method according to claim 30, wherein the serine/threonine kinase receptor is a type I receptor.

36. The method according to claim 30, wherein the serine/threonine kinase receptor is a type II receptor.

37. The method according to claim 30, wherein both type I and type II serine/threonine kinase receptors are administered.

38. The method according to claim 35, wherein the type I receptor is selected from the group consisting of ALK-1, ALK-2, ALK-3, ALK-4, ALK-5, ALK-6, and ALK-7.

39. The method according to claim 38, wherein the serine/threonine kinase receptor is selected from the group consisting of ALK-2, ALK-3, and ALK-6.

40. The method according to claim 30, wherein the serine/threonine kinase receptor is a recombinant ALK.

41. The method according to any one of claims 19-24, further comprising the step of administering to the target locus a bone morphogenic protein.

42. The method according to any one of claims 19-24, further comprising the step of administering to the target locus a nucleic acid encoding a bone morphogenic protein.

43. The method according to any one of claims 19-24, further comprising the step of administering to the target locus a vector comprising a nucleic acid encoding a bone morphogenic protein operably linked to an expression control sequence.

44. The method according to any one of claims 19-24, further comprising the step of administering to the target locus a cell comprising a vector comprising a nucleic acid encoding a bone morphogenic protein operably linked to an expression control sequence.

45. The method according to claim 41, wherein the bone morphogenic protein is selected from the groups consisting of OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, CDMP-3 (BMP-12), CDMP-2 (BMP-13), CDMP-1 (BMP-14), BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, dorsalin-1, DPP, Vg-1, Vgr-1, 60A protein, NODAL, UNIVIN, SCREW, ADMP, and NEURAL.

46. The method according to claim 45, wherein the bone morphogenic protein is OP-1.

47. The method according to any one of claims 19-24, wherein the tissue is selected from the group consisting of bone, cartilage, tendon, ligament and neural tissue.

48. The method according to claim 21 or 24, wherein the cell is a progenitor cell.

49. The method according to claim 48, wherein the progenitor cell is selected from the group consisting of an osteoprogenitor cell, a cartilage progenitor cell, a ligament progenitor cell, a tendon progenitor cell, and a neural progenitor cell.
